(19) United States
(12) Patent Application Publication
(10) Pub. No.: US 2022/0111123 A1
(43) Pub. Date: Apr. 14, 2022

(54) ENGINEERING A NATURALLY-DERIVED ADHESIVE AND CONDUCTIVE CARDIO-PATCH

(71) Applicant: THE REGENTS OF THE UNIVERSITY OF CALIFORNIA, Oakland, CA (US)

(72) Inventors: Nasim ANNABI, Los Angeles, CA (US); Brian Walker, Los Angeles, CA (US)

(21) Appl. No.: 17/602,985

(22) PCT Filed: Apr. 13, 2020
(86) PCT No.: PCT/US2020/027883

§ 371 Date: Oct. 11, 2021

Related U.S. Application Data
(60) Provisional application No. 62/832,502, filed on Apr. 11, 2019.

(51) Int. Cl. A61L 27/44; A61L 27/58; A61L 27/36; D01D 5/00; D01F 1/09

(52) U.S. Cl. CPC A61L 27/44; A61L 27/58; A61L 27/3687; H01B 1/122; D01D 5/0038; D01F 1/09; A61L 27/3695

(57) ABSTRACT

The present invention relates to adhesive and electroconductive cardiopatches designed to provide mechanical support and restore electromechanical coupling at the site of MI to minimize cardiac remodeling and preserve normal cardiac function.

### CROSS-REFERENCE TO RELATED APPLICATIONS

This application claims priority to U.S. Provisional Patent Application No. 62/832,502, filed Apr. 11, 2019, the contents of which is incorporated by reference herein in their entirety.

### STATEMENT REGARDING FEDERALLY SPONSORED RESEARCH OR DEVELOPMENT

This invention was made with government support under Grant No. R01-EB023052 and R01-HL140618 awarded by the National Institutes of Health (NIH). The government has certain rights in the invention.

### BACKGROUND OF THE INVENTION

Coronary heart disease (CHD) remains one of the major causes of death and disability in developed countries and accounts for approximately one third of all reported deaths in people older than 35 years of age (Sanchis-Gomar, F. et al., 2016, Ann Transl Med, 4(13):256). CHD often leads to partial or complete blockage of a coronary artery due to the rupture of an atherosclerotic plaque, in an event known as myocardial infarction (MI). MI severely restricts blood flow to the myocardium, which causes extensive cardiomyocyte (CM) death (Reis, L. A. et al., 2016, J Tissue Eng Regen Med, 10(1):11-28) and triggers a cascade of remodeling mechanisms such as left ventricle (LV) dilation, myocardium hypertrophy, and the appearance of fibrous and non-contractile scar tissue (Sutton, M. G. et al., 2000, Circulation, 101(25):2981-2988; Westman, P. C. et al., 2016, J Am Coll Cardiol, 67(17):2050-2060). Cardiac remodeling has a profound impact on both infarcted and non-infarcted regions of the heart, which greatly impairs normal cardiac function and could lead to chronic heart failure. Moreover, the formation of non-excitable and non-contractile scar tissue leads to asynchronous heart beating, owing to the interruption in the propagation of electrical impulses across the myocardium (Kai, D. et al., 2011, J Biomed Mater Res A, 99(3):376-385; Pfeffer, M. A. et al., 1990, Circulation, 81(4):1161-1172; Talman, V. et al., 2016, Cell Tissue Res, 365(3):563-581). In recent years, regenerative approaches based on multipotent and pluripotent stem cell therapy have shown great promise both in vitro and in vivo, albeit with highly heterogeneous outcomes and poor clinical translation (Cambria, E. et al., 2016, Transfus Med Hemother, 43(4):275-281; Le, T. Y. et al., 2017, Heart Lung Circ, 26(4):316-322).

Cardiac tissue engineering (TE) has enabled the development of temporary biomimetic scaffolds that can promote local cell growth and organization (Kai, D. et al., 2011, J Biomed Mater Res A, 99(3):376-385). These scaffolds are mainly aimed at providing mechanical support to the infarcted area, which minimizes cardiac remodeling and helps preserve the contractile function of the heart (Rai, R. et al., 2015, Adv Healthc Mater, 4(13): 2012-2025; Chen, Q. Z. et al., 2008, Materials Science and Engineering: R: Reports, 59(1):1-37; Malki, M. et al., 2018, Nano Lett). However, the recapitulation of the morphological and physiological features of the native myocardium remains challenging due to the complexity of structural, biochemical, and biophysical properties of the native cardiac microenvironment (Atmanli, A. et al., 2017, Trends Cell Biol, 27(5):352-364). For instance, these scaffolds should exhibit high durability and mechanical resilience to withstand repeated cycles of stretching during cardiac beating (Huyer, L. D. et al., 2015, Biomed Mater, 10(3):034004). Moreover, the composition of these cardiac patches should be based on biocompatible materials that can also be biodegraded in a clinically relevant time frame. Recent advancements in the field of material chemistry and microfabrication have allowed the engineering of a variety of cell-laden and acellular cardiac patches, which are based on both synthetic and naturally-derived biomaterials (Malki, M. et al., 2018, Nano Lett; Izadifar, M. et al., 2018, Tissue Eng Part C Meth, 24(2):74-88; Schaefer, J. A. et al., 2018, J Tissue Eng Regen Med, 12(2):546-556; Wang, Q. L. et al., 2017, J Cell Mol Med, 21(9):1751-1766; Tang, J. et al., 2017, Tissue Eng Part C Methods, 23(3):146-155; Sugiura, T. et al., 2016, J cardiothorac Surg, 11(1):163; Tallawi, M. et al., 2016, Mater Sci Eng C Mater Biol Appl, 69 (2016) 569-76). However, since electromechanical coupling is essential for the contractile function of the heart, alternative strategies to restore electrical conductivity at the site of MI should also be investigated (Monteiro, L. M. et al., 2017, NPJ Regen Med, 2:9).

Thus, there is a need in the field of tissue engineering to engineer scaffolds that can provide support to infarcted tissues and restore electromechanical coupling at the site of myocardial infarction to preserve cardiac function with minimal scar tissue formation. The present invention meets this need.

### SUMMARY OF THE INVENTION

In one aspect, the present invention provides a biocompatible conductive scaffold comprising: a fibrous biocompatible polymer conjugated to a first ionic constituent of a bio-ionic liquid (Bio-IL).

In one embodiment, the first ionic constituent of a Bio-IL is an organic quaternary amine. In one embodiment, the organic quaternary amine is choline. In one embodiment, the polymer is selected from the group consisting of: gelatin, elastin, elastin like polypeptides (ELP), collagen, hyaluronic acid (HA), tropoelastin, chitosan, alginate, poly(glycerol sebacate) (PGS), poly(ethylene glycol) (PEG), and poly(lactic acid) (PLA). In one embodiment, the biocompatible polymer and the first ionic constituent are conjugated via a diacrylate linker.

In one embodiment, the scaffold has a conductivity of at least about 0.23×10⁻¹±0.02×10⁻¹ siemens/meter (S/m). In one embodiment, the ratio of the biocompatible polymer to the first ionic constituent of a bio-ionic liquid (Bio-IL) is from about 1:4 to about 4:1 on a weight basis. In one embodiment, the scaffold is capable of supporting cell proliferation, tissue organization, and/or a function of an excitable cell. In one embodiment, the cell is selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte. In one embodiment, the scaffold is biodegradable. In one embodiment, the scaffold is seeded with a population of cells prior to implantation, the cells selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte.

In another aspect, the present invention provides a method of preparing a conductive scaffold, the method comprising the steps of: providing an ionic constituent of a bio-ionic liquid (Bio-IL) and a polymer; creating a fibrous mat using the polymer; placing the fibrous mat in a vacuum to remove excess solvent; placing the fibrous mat in a solution bath containing a photoinitiator; placing Bio-IL on the surface of the fibrous mat; and crosslinking the scaffold.

In one embodiment, the first ionic constituent of a Bio-IL is an organic quaternary amine. In one embodiment, the organic quaternary amine is choline. In one embodiment, the polymer is selected from the group consisting of: gelatin, elastin, elastin like polypeptides (ELP), collagen, hyaluronic acid (HA), tropoelastin, chitosan, alginate, poly(glycerol sebacate) (PGS), poly(ethylene glycol) (PEG), and poly(lactic acid) (PLA). In one embodiment, the polymer and the first ionic constituent of a Bio-IL are conjugated via a diacrylate linker.

In one embodiment, the scaffold has a conductivity of at least about 0.23×10⁻¹±0.02×10⁻¹ siemens/meter (S/m). In one embodiment, the ratio of the biocompatible polymer to the first ionic constituent of a Bio-IL is from about 1:4 to about 4:1 on a weight basis. In one embodiment, the scaffold is capable of supporting cell proliferation, tissue organization, and/or a function of an excitable cell. In one embodiment, the cell is selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte. In one embodiment, the scaffold is biodegradable. In one embodiment, the crosslinking step is performed for between about 100 and 500 seconds. In one embodiment, the crosslinking step is performed using UV irradiation or visible light. In one embodiment, the crosslinking step is performed on both side of the scaffold. In one embodiment, the method further comprises a step of seeding cells on the scaffold, the cells selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1H depict synthesis and physical properties of electrospun GelMA/Bio-IL cardiopatches. FIG. 1A depicts a schematic of the electrospinning of GelMA fibrous mats followed by soaking in Irgacure solution and Bio-IL addition prior to photocrosslinking with UV light for 5 min to form patches. Representative SEM images of patches formed by using 10% (w/v) GelMA with FIG. 1B 0%, and FIG. 1C) 33% (v/v) Bio-IL. FIG. 1D depicts the electrical conductivity of cardiopatches fabricated with varying GelMA and Bio-IL concentrations, showing that the electrical conductivity of patches increased concomitantly when fabricated with higher concentrations of Bio-IL. FIG. 1E depicts the electroconductive properties of cardiopatches after incubation in DPBS at 37° C. for 2 and 4 days, which demonstrated that electrical conductivity did not decrease. FIG. 1F depicts swelling ratio, FIG. 1G depicts degradation rate in collagenase type II solution over time, and FIG. 1H depicts elastic modulus of fabricated cardiopatches (for swelling ratio and degradation test 10% (w/v) GelMA was used). Error bars indicate standard error of the means, asterisks mark significance levels of p<0.05 (*), p<0.01 (**), and p<0.001 (***).

FIG. 2A through FIG. 2H depict ex vivo adhesive properties and electrical conductivity of GelMA/Bio-IL cardiopatches. FIG. 2A depicts representative image of a GelMA/Bio-IL cardiopatch photocrosslinked on explanted rat heart demonstrating the high adhesion of the cardiopatch (red arrows) to cardiac tissues. FIG. 2B depicts standard wound closure test using explanted rat heart as the biological substrate to test the adhesion strength of GelMA/Bio-IL cardiopatches. FIG. 2C depicts quantification of the adhesion strength exhibited by cardiopatches fabricated with 10% (w/v) GelMA and varying concentrations of Bio-IL on explanted rat hearts. Cardiopatches fabricated with higher concentrations of Bio-IL demonstrated higher adhesion strength to cardiac tissue.

FIG. 2D depicts representative images of GelMA/Bio-IL cardiopatch fabricated with 10% (w/v) GelMA and 66% (v/v) Bio-IL photocrosslinked on the defect site of an explanted rat heart to measure the burst pressure. FIG. 2E depicts quantification of the burst pressure of GelMA/Bio-IL cardiopatches formed with varying concentrations of Bio-IL and photocrosslinked on the defect site of rat heart showed no significant difference when compared to the burst pressure of a healthy rat heart. FIG. 2F depicts H&E staining of cardiopatch-tissue interfaces. The tight interface indicates a strong bonding of the GelMA/Bio-IL cardiopatch to the murine myocardium. The schematic in FIG. 2F showing the electrostatic forces between positively charged Bio-IL and negatively charged surface of cardiac muscle tissue and cells, as well as covalent bonds between methacrylate groups of GelMA and NH₂ functional groups in cardiac tissue. These two types of bonding led to strong adhesion. FIG. 2G depicts schematic of ex vivo abdominal tissue placed adjacently on GelMA/Bio-IL cardiopatches fabricated with 10% (w/v) and varying concentrations of Bio-IL to determine the threshold voltage needed to stimulate both sections of abdominal tissue. FIG. 2H depicts quantification of the threshold voltage of GelMA/Bio-IL cardiopatches significantly decreased for patches fabricated with 100% (v/v) Bio-IL compared to those fabricated with 33% (v/v) Bio-IL demonstrating enhanced electrical properties with higher concentrations of Bio-IL. Error bars indicate standard error of the means, asterisks mark significance levels of p<0.05 (*).

FIG. 3A through FIG. 3I depict 2D co-cultures of CMs and CFs on GelMA/Bio-IL cardiopatches. The in vitro cytocompatibility of the engineered cardiopatches was evaluated using 2D co-cultures of freshly isolated CMs and CFs (ratio 2:1) growing on cardiopatches fabricated with different concentrations of Bio-IL. FIG. 3A depicts representative Live/Dead images of CMs/CFs growing on patches containing 0% and 66% (v/v) Bio-IL at day 7 post-seeding. FIG. 3B depicts representative actin/DAPI images of CMs/CFs growing on patches fabricated with 0% and 66% (v/v) Bio-IL at day 7 post-seeding (Scale bar=200 μm). Bar graphs showing the quantification of FIG. 3C cell viability, and FIG. 3D metabolic activity of 2D co-cultures of CMs/CFs at days 1, 4, and 7 post-seeding, growing on cardiopatches engineered with different concentrations of Bio-IL. FIG. 3E depicts characterization of the beating frequency (beats/min) of CMs/CFs throughout 7 days of culture growing on cardiopatches fabricated with varying concentrations of Bio-IL. Representative immunofluorescent images of CMs/CFs at day 7 post-seeding growing on the surface of patches containing FIG. 3F 0%, and FIG. 3G 66% (v/v) Bio-IL (green: sarcomeric α-actinin, red: connexin 43, blue: DAPI) (Scale bar=50 μm). Quantification of the relative levels of expression (i.e., intensity of fluorescence) of FIG. 3H connexin 43, and FIG. 3I sarcomeric α-actinin in co-cultures of CMs/CFs on engineered patches at day 7 post-seeding.

FIG. 4A through FIG. 4F depict in vivo evaluation of GelMA/Bio-IL cardiopatches using a murine model of MI. Experimental MIs were induced via permanent ligation of the LAD coronary artery. FIG. 4A depicts representative images showing: FIG. 4A (i) depicts LAD ligation (white circle), FIG. 4A (ii) depicts photocrosslinking of cardiopatches (white arrows) using UV light, FIG. 4A (iii) depicts photocrosslinked cardiopatch on the heart, and FIG. 4A (iv) depicts excised whole heart with cardiopatch distal to the site of LAD ligation after 21 days. Representative Masson's trichrome stained images from the interface between FIG. 4B depicts GelMA and FIG. 4C depicts GelMA/Bio-IL cardiopatches after 21 days (Scale bar=400 μm). Representative Masson's trichrome and fluorescent stained images of excised hearts showing the location of the MIs in FIG. 4D (i-iii) depict untreated animal (sham), and animals treated with FIG. 4E (i-iii) depict pristine GelMA patches, and FIG. 4F (i-iii) depict GelMA/Bio-IL cardiopatches

FIG. 5A through FIG. 5E depict characterization of proposed reaction between electrospun GelMA and Bio-IL to synthesize cardiac patches. FIG. 5A depicts H-NMR analysis of acrylated choline-based Bio-IL. FIG. 5B depicts GelMA prepolymer solution, FIG. 5C depicts photocrosslinked GelMA, and FIG. 5D depicts photocrosslinked GelMA/Bio-IL patches. FIG. 5E depicts the degree of crosslinking of the GelMA/Bio-IL cardiopatches was significantly greater compared with the pristine GelMA patches.

FIG. 6A through FIG. 6B depict characterization of the fiber size of GelMA/Bio-IL cardiopatches. FIG. 6A depicts representative SEM images of electrospun cardiopatches synthesized with 10% (w/v) GelMA and 0%, 33%, 66%, and 100% (v/v) Bio-IL. FIG. 6B depicts quantification of fiber diameter, demonstrating that fiber size did not significantly change by varying the Bio-IL concentration.

FIG. 7A through FIG. 7B depict electrical properties of cardiopatches. FIG. 7A depicts schematic of two-probe electrical station used to characterize the conductive properties of cardiopatches. FIG. 7B depicts the electrical conductivity of cardiopatches in relaxed position compared to the conductivity of patches stretched to 20%, and 40% strain. Cardiopatches at 40% strain rate exhibit no significant change in electrical conductivity compared to patches under 0% strain

FIG. 8 depicts swelling ratio of engineered cardiopatches. The in vitro swelling ratio for cardiopatches fabricated with 15% GelMA and different concentrations of Bio-IL. Error bars indicate standard error of the means, asterisks mark significance levels of p<0.05 (*), p<0.01 (**), and p<0.001 (***).

FIG. 9A through FIG. 9B depict degradation of engineered cardiopatches in DPBS. The in vitro degradation rate for cardiopatches fabricated with (FIG. 9A) 10% or (FIG. 9B) 15% (w/v) GelMA, and varying concentrations of Bio-IL. Error bars indicate standard error of the means, asterisks mark significance levels of p<0.05 (*), p<0.01 (**), and p<0.001 (***).

FIG. 10A through FIG. 10C depict mechanical properties of cardiopatches. FIG. 10A depicts representative images of tensile test conducted on cardiopatches formed by using 10% (w/v) GelMA and 66% (v/v) Bio-IL. The FIG. 10B ultimate strain and FIG. 10C ultimate stress of GelMA/Bio-IL cardiopatches fabricated using various concentrations of GelMA and Bio-IL. Error bars indicate standard error of the means, asterisks mark significance levels of p<0.05 (*), p<0.01 (**), and p<0.001 (***).

FIG. 11 A through FIG. 11D depict in vitro adhesion strength of GelMA/Bio-IL cardiopatches. FIG. 11A depicts schematic of wound closure test on cardiopatches using porcine skin. FIG. 11B depicts patches fabricated with 10% (w/v) GelMA and varying concentrations of Bio-IL displayed an increasing adhesion strength to porcine skin when fabricated with increasing concentration of Bio-IL. Additionally, these GelMA/Bio-IL cardiopatches showed a significantly higher adhesion strength when compared with commercially available tissue sealants, such as Coseal™, and Evicel®. FIG. 11C depicts schematic of the measurement of the burst pressure of cardiopatches. FIG. 11D depicts cardiopatches fabricated with 10% (w/v) GelMA and varying concentrations of Bio-IL exhibited an increasing burst pressure strength when fabricated with an increasing concentration of Bio-IL. The burst pressure strength of GelMA/Bio-IL cardiopatches was also greater than Coseal™, and Evicel®. Error bars indicate standard error of the means, asterisks mark significance levels of p<0.05 (*), p<0.01 (**), and p<0.001 (***).

FIG. 12A through FIG. 12B depict in vitro evaluation of cell proliferation. FIG. 12A depicts representative fluorescent micrographs of CM/CF co-cultures using F-actin/DAPI staining. FIG. 12B depicts quantification of cell proliferation (cells/mm²).

FIG. 13 depicts coaxial electrospinning set up to form GelMA mats containing SDF-1 in core and VEGF in shell.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the field of surgical devices, including those indicated for the treatment of peripheral nerve anastomosis. Those of ordinary skill in the art may recognize that other elements and/or steps are desirable and/or required in implementing the present invention. However, because such elements and steps are well known in the field, and because they do not facilitate a better understanding of the present invention, a discussion of such elements and steps is not provided herein. The disclosure herein is directed to all such variations and modifications to such elements and methods known to those skilled in the art.

### Definitions

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, a “culture,” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “monolayer” by producing glycoprotein-like material that causes the cells to adhere to the plastic or glass of the Petri dish. A layer of culture medium, containing nutrients suitable for cell growth, is then placed on top of the monolayer, and the culture is incubated to promote the growth of the cells.

“Differentiation medium” is used herein to refer to a cell growth medium comprising an additive or a lack of an additive such that a stem cell or progenitor cell, that is not fully differentiated, develops into a cell with some or all of the characteristics of a differentiated cell when incubated in the medium.

As used herein, a “bio-ionic liquid” refers to a salt that has a melting temperature below room temperature (e.g., the melting temperature is less than 10° C., less than 15° C., less than 20° C., less than 25° C., less than 30° C., or less than 35° C.) and that contains a cation and an anion, at least one of which is a biomolecule (i.e., a molecule found in a living organism) or a biocompatible organic molecule. Examples of bio-ionic liquids are organic salts of choline, such as carboxylate salts of choline, choline bicarbonate, choline maleate, choline succinate, and choline propionate. An ionic constituent of a bio-ionic liquid is a cation or anion component of a bio-ionic liquid. Examples of ionic constituents of bio-ionic liquids for use in the invention are biocompatible organic cations such as choline and other biocompatible quaternary organic amines, as well as biocompatible organic anions such as carboxylic acids, including formate, acetate, propionate, butyrate, malate, succinate, citrate, and the like.

The term “electroprocessing” as used herein shall be defined broadly to include all methods of electrospinning, electrospraying, electroaerosoling, and electrosputtering of materials, combinations of two or more such methods, and any other method wherein materials are streamed, sprayed, sputtered or dripped across an electric field and toward a target. The electroprocessed material can be electroprocessed from one or more grounded reservoirs in the direction of a charged substrate or from charged reservoirs toward a grounded target. “Electrospinning” means a process in which fibers are formed from a solution or melt by streaming an electrically charged solution or melt through an orifice. “Electroaerosoling” means a process in which droplets are formed from a solution or melt by streaming an electrically charged polymer solution or melt through an orifice. The term electroprocessing is not limited to the specific examples set forth herein, and it includes any means of using an electrical field for depositing a material on a target.

As used herein, “extracellular matrix composition” includes both soluble and non-soluble fractions or any portion thereof. The non-soluble fraction includes those secreted ECM proteins and biological components that are deposited on the support or scaffold. The soluble fraction includes refers to culture media in which cells have been cultured and into which the cells have secreted active agent(s) and includes those proteins and biological components not deposited on the scaffold. Both fractions may be collected, and optionally further processed, and used individually or in combination in a variety of applications as described herein.

As used herein, a “graft” refers to a cell, tissue, organ, or biomaterial that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft,” “allogeneic transplant,” “allogeneic implant,” and “allogeneic graft.” A graft from an individual to his identical twin is referred to herein as an “isograft,” a “syngeneic transplant,” a “syngeneic implant” or a “syngeneic graft.” A “xenograft,” “xenogeneic transplant,” or “xenogeneic implant” refers to a graft from one individual to another of a different species. The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, “polymer” includes copolymers. “Copolymers” are polymers formed of more than one polymer precursor. Polymers as used herein include those that are soluble in a solvent and are insoluble in an antisolvent.

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, “tissue engineering” refers to the process of generating a tissue ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein, the terms “tissue grafting” and “tissue reconstructing” both refer to implanting a graft into an individual to treat or alleviate a tissue defect, such as a lung defect or a soft tissue defect.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Scaffolds

The present invention provides a new class of adhesive and electroconductive electrospun fibrous scaffold patches. The scaffolds can be used as cardiopatches for the treatment of myocardial infarction (MI). The scaffolds are useful for engineering tissues with high adhesive strength and tunable mechanical and conductive properties. Incorporation of bio-ionic liquid (Bio-IL) into the electroprocessed network provides tunable electroconductive properties to the Bio-IL conjugated engineered scaffolds.

In some embodiments, the scaffold of this invention is biocompatible and biodegradable with tunable conductivity. The scaffold includes a biocompatible polymer conjugated to an ionic constituent of a bio-ionic liquid via a linker. The linker is a chemical moiety that covalently binds the constituent of a bio-organic liquid to the biocompatible polymer and is biocompatible and biodegradable. Suitable linkers include diacrylates, disulfides, esters, and the like.

In some embodiments, the scaffold of this invention can include one or more of the following features. The ionic constituent of a bio-ionic liquid can be, for example, choline or another quaternary amine. In certain embodiments, the ionic constituent is another cationic constituent of a bio-ionic liquid. In certain embodiments, the ionic constituent is an anionic constituent of a bio-ionic liquid. The polymer can be any biocompatible polymer, such as a polymer found in a living organism, from which a conjugate is formed by the covalent attachment of an ionic constituent of a bio-organic liquid through a linker moiety. For example, the polymer can be gelatin, elastin, one or more elastin-like polypeptides (ELP), collagen (any type of collagen or a mixture thereof), hyaluronic acid (HA), alginate, poly(glycerol sebacate) (PGS), or poly(ethylene glycol) (PEG).

In some embodiments, the scaffold has a conductivity that is at least about 0.23×10⁻¹±0.02×10⁻¹ siemens/meter (S/m). In some embodiments, the conductivity of the scaffold can be as high as 1.9×10⁻ⁱ±0.18×10⁻¹ S/m. The ratio of the polymer to the ionic constituent can range, for example, from 100:0 to 1:4 by weight; i.e., the weight percentage of the ionic constituent of a bio-ionic liquid can range from 0 wt % (or a small value >0 wt %, e.g., 0.1 wt %) to about 80 wt %. The conjugated polymer can be present at, for example, from 10% to 20% of the weight of the scaffold, or from 11% to 20%, or 12% to 20%, or 15% to 20%, or about 10%, about 11%, about 12%, about 13%, about 14%, about 15%, about 16%, about 17%, about 18%, about 19%, or about 20% (all wt %). Alternatively, the conjugated polymer can be present at from about 20 wt % to about 80 wt % of the scaffold. Additionally, the conductivity may be tuned by changing the ratio of the polymer to the ionic constituent of the Bio-IL. The conductivity may be tuned also by changing the percent weight of the total polymer in the scaffold.

In some embodiments, the scaffold has an elastic modulus that is between about 8.76±0.42 kPa to 145.50±4.10 kPa. In some embodiments, the elastic modulus of the scaffold may be tuned by changing the ratio of the polymer to the Bio-IL. The elastic modulus of the scaffold may be tuned also by changing the percent weight of the total polymer in the scaffold. The porosity and the swellability of the scaffold may be tuned by changing the ratio of the polymer to the Bio-IL or by changing the percent weight of the total polymer in the scaffold. In some embodiments, the scaffold is capable of supporting cell proliferation, organization, and/or function of an excitable cell in both 2D cell seeding and 3D cell encapsulation. The cell type, for example, can be a nerve cell, a muscle cell, a fibroblast, a preosteoblast, an endothelial cell, or a mesenchymal stem cell. In some embodiments, the muscle cell is a cardiomyocyte.

In some embodiments, the scaffold of this invention is a temporary scaffold for cells that supports electroactive modulation of the cells.

Embodiments of the scaffolds of the present invention can have one or more of the following features. The scaffold can support one or more of adhesion, proliferation, migration, and differentiation of cells. These cells may be excitable cells, e.g., neurons, cardiomyocytes, fibroblasts, preosteoblasts, endothelial cells, or mesenchymal stem cells.

According to a further aspect of the invention a method of preparing a conductive scaffold is provided. The method includes: (a) providing an ionic constituent of a Bio-IL and a polymer, (b) creating a fibrous mat using the polymer, (c) removing any remaining solvent by placing the fibrous mat in vacuum, (d) placing the fibrous mat in a solution bath containing a photoinitiator, (e) placing Bio-IL on the surface of fibrous mats, and (f) crosslinking the scaffold using UV irradiation for between about 100 and 500 seconds on each side of the scaffold.

In one embodiment, the above method can include one or more of the following features. The Bio-IL ionic constituent can be choline. The polymer can be poly(ethylene) glycol. The modified polymer can be poly(ethylene glycol) diacrylate. Alternatively, the polymer can be gelatin. The modified polymer can be gelatin methacryloyl photoinitiator can be Eosin Y caprolactone (VC), triethanolamine (TEOA) (for visible light), or Irgacure 2959 (for UV). In some embodiments, the photoinitiator produces free radicals when exposed to ultraviolet (UV) or visible light. In some embodiments, photoinitiators include 1-[4-(2-hydroxyethoxy)-phenyl]-2-hydroxy-2-methyl-1-propane-1-one (Irgacure 2959, BASF, Florham Park, N.J., USA), azobisisobutyronitrile, benzoyl peroxide, di-tert-butyl peroxide, 2,2-dimethoxy-2-phenylacetophenone, Eosin Y, etc. In some embodiments, the photoinitiator is 1-[4-(2-hydroxyethoxy)-phenyl]-2-hydroxy-2-methyl-1-propane-1-one.

In some embodiments, the visible light activated photoinitiator is selected from the group consisting of: Eosin Y, triethanolamine, vinyl caprolactam, dl-2,3-diketo-1,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide (Ir819), 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane (IVOCERIN), derivatives thereof, and any combination thereof.

The exemplary scaffolds and methods of the present invention provide several advantages. Scaffolds with different biomechanical and electroconductive profiles can be generated by varying the polymer to Bio-IL ratio and the concentration of the total Bio-IL conjugated polymer in the scaffolds. In other words, the biomechanical and electroconductive properties of the scaffolds are tunable. Further, the engineered scaffolds are biodegradable and elicit minimal inflammatory responses.

In some embodiments, the scaffold can have any suitable shape. In some embodiments, the scaffolds are substantially planar, such as in the form of a sheet. In other embodiments, the scaffolds can be shaped into a three-dimensional structure, such as a tube or a sphere. The scaffolds can have any suitable thickness, such as a thickness that is less than 100 μm or as great as several millimeters. In some embodiments, the thickness of the scaffolds is between about 500 μm to about 2000 μm or about 5000 μm. In various embodiments, the scaffolds can be trimmed or sized to accommodate any suitable shape.

In various embodiments, the scaffolds can be modified with one or more functional groups for covalently attaching a variety of proteins (e.g., collagen) or compounds such as therapeutic agents. Therapeutic agents which may be linked to the scaffold include, but are not limited to, analgesics, anesthetics, antifungals, antibiotics, anti-inflammatories, anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the scaffold may be via a protease sensitive linker or other biodegradable linkage. Molecules which may be incorporated into the biomimetic scaffold include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents.

In various embodiments, the scaffolds can further comprise one or more polysaccharides, including glycosaminoglycans (GAGs) or glucosaminoglycans, with suitable viscosity, molecular mass, and other desirable properties. The term “glycosaminoglycan” is intended to encompass any glycan (i.e., polysaccharide) comprising an unbranched polysaccharide chain with a repeating disaccharide unit, one of which is always an amino sugar. These compounds as a class carry a high negative charge, are strongly hydrophilic, and are commonly called mucopolysaccharides. This group of polysaccharides includes heparin, heparan sulfate, chondroitin sulfate, dermatan sulfate, keratan sulfate, and hyaluronic acid. These GAGs are predominantly found on cell surfaces and in the extracellular matrix. The term “glucosaminoglycan” is also intended to encompass any glycan (i.e. polysaccharide) containing predominantly monosaccharide derivatives in which an alcoholic hydroxyl group has been replaced by an amino group or other functional group such as sulfate or phosphate. An example of a glucosaminoglycan is poly-N-acetyl glucosaminoglycan, commonly referred to as chitosan. Exemplary polysaccharides that may be useful in the present invention include dextran, heparan, heparin, hyaluronic acid, alginate, agarose, carageenan, amylopectin, amylose, glycogen, starch, cellulose, chitin, chitosan and various sulfated polysaccharides such as heparan sulfate, chondroitin sulfate, dextran sulfate, dermatan sulfate, or keratan sulfate.

In various embodiments, the scaffolds can further comprise one or more extracellular matrix materials and/or blends of naturally occurring extracellular matrix materials, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. The scaffolds can further comprise one or more carbohydrates such as chitin, chitosan, alginic acids, and alginates such as calcium alginate and sodium alginate. These materials may be isolated from plant products, humans or other organisms or cells or synthetically manufactured. Also contemplated are crude extracts of tissue, extracellular matrix material, or extracts of non-natural tissue, alone or in combination. Extracts of biological materials, including but are not limited to cells, tissues, organs, and tumors may also be included.

In various embodiments, the scaffolds can further comprise one or more synthetic materials. The synthetic materials can be biologically compatible for administration in vivo or in vitro. Such polymers include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Polymers with cationic moieties can also be used, such as poly(allyl amine), poly(ethylene imine), poly(lysine), and poly(arginine). The polymers may have any molecular structure including, but not limited to, linear, branched, graft, block, star, comb, and dendrimer structures.

In some embodiments, the scaffolds can further comprise one or more natural or synthetic drugs, such as nonsteroidal anti-inflammatory drugs (NSAIDs). In one embodiment, the scaffolds can further comprise antibiotics, such as penicillin. In one embodiment, the scaffolds can further comprise natural peptides, such as glycyl-arginyl-glycyl-aspartyl-serine (GRGDS), arginylglycylaspartic acid (RGD), and amelogenin. In one embodiment, the scaffolds can further comprise proteins, such as chitosan and silk. In one embodiment, the scaffolds can further comprise sucrose, fructose, cellulose, or mannitol. In one embodiment, the scaffolds can further comprise extracellular matrix proteins, such as fibronectin, vitronectin, laminin, collagens, and vixapatin (VP12). In one embodiment, the scaffolds can further comprise disintegrins, such as VLO4. In one embodiment, the scaffolds can further comprise decellularized or demineralized tissue. In one embodiment, the scaffolds can further comprise synthetic peptides, such as emdogain. In one embodiment, the scaffolds can further comprise nutrients, such as bovine serum albumin. In one embodiment, the scaffolds can further comprise vitamins, such as vitamin B2, vitamin Ad, Vitamin D, Vitamin E, and Vitamin K. In one embodiment, the scaffold can further comprise nucleic acids, such as mRNA and DNA. In one embodiment, the scaffolds can further comprise natural or synthetic steroids and hormones, such as dexamethasone, hydrocortisone, estrogens, and its derivatives. In one embodiment, the scaffold can further comprise growth factors, such as fibroblast growth factor (FGF), transforming growth factor beta (TGF-β), and epidermal growth factor (EGF). In one embodiment, the scaffolds can further comprise a delivery vehicle, such as nanoparticles, microparticles, liposomes, viral and non-viral transfection systems.

In one embodiment, the scaffolds are provided cell-free. In another embodiment, the scaffolds are provided pre-seeded with one or more populations of cells to form an artificial tissue construct. The cells can be cultured in any suitable environment, including under in vivo and in vitro conditions. Non-limiting examples of suitable cells include nerve cells, muscle cells, cardiomyocytes, fibroblasts, preosteoblasts, endothelial cells, mesenchymal stem cells, pluripotent stem cells, embryonic stems cells, hematopoietic stem cells, adipose derived stem cells, bone marrow derived stem cells, osteocytes, epithelial cells, neurocytes, and the like.

The artificial tissue construct may be autologous, where the cell populations are derived from a patient's own tissue, or allogenic, where the cell populations are derived from another subject within the same species as the patient. The artificial organ construct may also be xenogenic, where the different cell populations are derived form a mammalian species that is different from the subject. For example the cells may be derived from organs of mammals such as humans, monkeys, dogs, cats, mice, rats, cows, horses, pigs, goats and sheep.

Cells may be isolated from a number of sources, including, for example, biopsies from living subjects and whole-organ recover from cadavers. The isolated cells can be autologous cells, obtained by biopsy from the subject intended to be the recipient. The biopsy may be obtained using a biopsy needle, a rapid action needle which makes the procedure quick and simple.

Cells may be isolated using techniques known to those skilled in the art. For example, the tissue may be disaggregated mechanically and/or treated with digestive enzymes and/or chelating agents that weaken the connections between neighboring cells making it possible to disperse the tissue into a suspension of individual cells without appreciable cell breakage. Enzymatic dissociation may be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase and dispase. Mechanical disruption may also be accomplished by a number of methods including, but not limited to, scraping the surface of the tissue, the use of grinders, blenders, sieves, homogenizers, pressure cells, or sonicators.

Once the tissue has been reduced to a suspension of individual cells, the suspension may be fractionated into subpopulations from which the cells elements may be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting.

Cell fractionation may also be desirable, for example, when the donor has diseases such as cancer or metastasis of other tumors to the desired tissue. A cell population may be sorted to separate malignant cells or other tumor cells from normal noncancerous cells. The normal noncancerous cells, isolated from one or more sorting techniques, may then be used for tissue reconstruction.

Isolated cells may be cultured in vitro to increase the number of cells available for seeding the biomimetic scaffold. The use of allogenic cells, such as autologous cells, can be used to prevent tissue rejection. However, if an immunological response does occur in the subject after implantation of the artificial organ, the subject may be treated with immunosuppressive agents such as cyclosporin or FK506 to reduce the likelihood of rejection. In certain embodiments, chimeric cells, or cells from a transgenic animal, may be seeded onto the biocompatible scaffold.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chances of rejection by the host. In addition, transfection could also be used for gene delivery.

Isolated cells may be normal or genetically engineered to provide additional or normal function. Methods for genetically engineering cells with retroviral vectors, polyethylene glycol, or other methods known to those skilled in the art may be used. These include using expression vectors which transport and express nucleic acid molecules in the cells. (See Goeddel; Gene Expression Technology: Methods in Enzymology 185, Academic Press, San Diego, Calif. (1990). Vector DNA may be introduced into prokaryotic or cells via conventional transformation or transfection techniques. Suitable methods for transforming or transfecting host cells can be found in Sambrook et al. (Molecular Cloning: A Laboratory Manual, 3nd Edition, Cold Spring Harbor Laboratory press (2001)), and other laboratory textbooks.

Seeding of cells onto the scaffolds may be performed according to standard methods. For example, the seeding of cells onto polymeric substrates for use in tissue repair has been reported (see, e.g., Atala, A. et al., J. Urol. 148(2 Pt 2): 658-62 (1992); Atala, A., et al. J. Urol. 150 (2 Pt 2): 608-12 (1993)). Cells grown in culture may be trypsinized to separate the cells, and the separated cells may be seeded on the scaffolds. Alternatively, cells obtained from cell culture may be lifted from a culture plate as a cell layer, and the cell layer may be directly seeded onto the scaffolds without prior separation of the cells.

In some embodiments, a range of 1 million to 50 million cells are suspended in medium and applied to each square centimeter of a surface of a scaffold. The scaffold is incubated under standard culturing conditions, such as, for example, 37° C. 5% CO₂, for a period of time until the cells become attached. However, it will be appreciated that the density of cells seeded onto the scaffold may be varied. For example, greater cell densities promote greater tissue regeneration by the seeded cells, while lesser densities may permit relatively greater regeneration of tissue by cells infiltrating the graft from the host. Other seeding techniques may also be used depending on the matrix or scaffold and the cells. For example, the cells may be applied to the matrix or scaffold by vacuum filtration. Selection of cell types, and seeding of cells onto a scaffold, will be routine to one of ordinary skill in the art in light of the teachings herein.

In some embodiments, the scaffolds are seeded with one population of cells to form an artificial tissue construct. In another embodiment, the scaffolds are seeded on two sides with two different populations of cells. This may be performed by first seeding one side of a scaffold and then seeding the other side. For example, the scaffold may be placed with one side on top and seeded. The scaffold may then be repositioned so that a second side is on top. The second side may then be seeded with a second population of cells. Alternatively, both sides of the scaffold may be seeded at the same time. For example, two cell chambers may be positioned on both sides (i.e., a sandwich) of the scaffold. The two chambers may be filled with different cell populations to seed both sides of the scaffold simultaneously. The sandwiched scaffold may be rotated or flipped frequently to allow equal attachment opportunity for both cell populations.

In another embodiment, two separate scaffolds may be seeded with different cell populations. After seeding, the two scaffolds may be attached together to form a single scaffold with two different cell populations on the two sides. Attachment of the scaffolds to each other may be performed using standard procedures such as fibrin glue, liquid co-polymers, sutures, and the like.

In order to facilitate cell growth on the scaffold of the present invention, the scaffold may be coated with one or more cell adhesion-enhancing agents. These agents include but are not limited to collagen, laminin, and fibronectin. The scaffold may also contain cells cultured on the scaffold to form a target tissue substitute. In the alternative, other cells may be cultured on the scaffold of the present invention.

### Fabrication of Scaffolds

As described elsewhere herein, the scaffolds of the present invention can be fabricated using electrospinning. Electrospinning is a fiber forming technique that relies on charge separation to produce nano- to microscale fibers, which typically form a non-woven matrix. The terms “nonwoven matrix”, “nonwoven mesh” or “nonwoven scaffold” are used interchangeably herein to refer to a material comprising a randomly interlaced fibrous web of fibers. Generally, individual electrospun fibers have large surface-to-volume and high aspect ratios resulting from the smallness of their diameters. These beneficial properties of the individual fibers are further enhanced by the porous structure of the non-woven fabric, which allows for cell infiltration, cell aggregation, and tissue formation.

The electrospinning process is affected by varying the electric potential, flow rate, solution concentration, capillary-collector distance, diameter of the needle, and ambient parameters like temperature. Therefore, it is possible to manipulate the porosity, surface area, fineness and uniformity, diameter of fibers, and the pattern thickness of the matrix.

Electrospinning is an atomization process of a fluid which exploits the interactions between an electrostatic field and the fluid. That is, electrospinning is a method of electrostatic extrusion used to produce sub-micron sized fibers. In one aspect, the fluid can be a conducting fluid. Also known within the fiber forming industry as electrostatic spinning, the process of electrospinning generally involves the creation of an electrical field at the surface of a liquid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The resulting electrical forces create a jet of liquid which carries electrical charge. Thus, the liquid jets may be attracted to other electrically charged objects at a suitable electrical potential. As the jet of liquid elongates and travels, it will harden and dry. Fibrils of nanometer-range diameter can be produced. The hardening and drying of the elongated jet of liquid may be caused by cooling of the liquid, by evaporation of a solvent, or by a curing mechanism. The produced fibers are collected on a suitably located, oppositely charged receiver and subsequently removed from it as needed, or directly applied to an oppositely charged generalized target area.

Fibers can be electrospun from high viscosity polymer melts or polymers dissolved in volatile solvents; the end result is a non-woven mesh of fiber. Solution viscosity can be controlled by modifying polymer concentration, molecular weight, and solvents. Electric field properties can be controlled by modifying bias magnitude or tip-to-target distance. Polymers can be co-spun from same the solution and the polymer phase can be selectively removed. Further, fibers can be electrospun from a multiphasic polymer solution or from an emulsion. For example, polyurethane fibers can be electrospun from a multiphasic polyurethane solution. Emulsifying the solution can increase the solution viscosity, thereby inducing fiber formation at lower concentrations. The resultant fibers can be created having diameters as a function of aqueous content.

A broad range of polymers can be used in electrospinning the scaffolds, including polyamides, polylactides, cellulose derivatives, water soluble polymers such as polyethyleneoxide, as well as polymer blends or polymers containing solid nanoparticles or functional small molecules. The scaffolds can also be fabricated with numerous synthetic biodegradable polymers, such as poly(ε-caprolactone) (PCL), poly(lactic acid) (PLA), poly(glycolic acid) (PGA), the copolymers poly(lactide-co-glycolide) (PLGA), and poly(L-lactide-co-ε-caprolactone) [P(LLA-CL)].

In the most fundamental sense, the electrospinning apparatus for electrospinning material includes an electrodepositing mechanism and a target substrate. The electrodepositing mechanism includes a reservoir or reservoirs to hold the one or more solutions that are to be electrospun or electrodeposited. The reservoir or reservoirs have at least one orifice or nozzle to allow the streaming of the solution from the reservoirs. One or a plurality of nozzles may be configured in an electrospinning apparatus. If there are multiple nozzles, each nozzle is attached to one or more reservoirs containing the same or different solutions. Similarly, there can be a single nozzle that is connected to multiple reservoirs containing the same or different solutions. Multiple nozzles may be connected to a single reservoir. Because different embodiments involve single or multiple nozzles and/or reservoirs, any references herein to one or nozzles or reservoirs should be considered as referring to embodiments involving single nozzles, reservoirs, and related equipment as well as embodiments involving plural nozzles, reservoirs, and related equipment. The size of the nozzles can be varied to provide for increased or decreased flow of solutions out of the nozzles. One or more pumps used in connection with the reservoirs can be used to control the flow of solution streaming from the reservoir through the nozzle or nozzles. The pump can be programmed to increase or decrease the flow at different points during electrospinning.

The electrospinning occurs due to the presence of a charge in either the orifices or the target, while the other is grounded. In some embodiments, the nozzle or orifice is charged and the target is shown to be grounded. Those having skill in the electrospinning arts will recognize that the nozzle and solution can be grounded and the target can be electrically charged. The creation of the electrical field and the effect of the electrical field on the electroprocessed materials or substances that will form the electroprocessed composition.

Any solvent can be used that allows delivery of the material or substance to the orifice, tip of a syringe, or other site from which the material will be electroprocessed. The solvent may be used for dissolving or suspending the material or the substance to be electroprocessed. Solvents useful for dissolving or suspending a material or a substance depend on the material or substance. Electrospinning techniques often require more specific solvent conditions. For example, polyurethane can be electrospun as a solution or suspension in water, 2,2,2-trifluoroethanol, 1,1,1,3,3,3-hexafluoro-2-propanol (also known as hexafluoroisopropanol or HFIP), or combinations thereof. Alternatively, polyurethane can be electrospun from solvents such as urea, monochloroacetic acid, water, 2,2,2-trifluoroethanol, HFIP, or combinations thereof. Other lower order alcohols, especially halogenated alcohols, may be used. Additional solvents that may be used or combined with other solvents include acetamide, N-methylformamide, N,N-dimethylformamide (DMF), dimethylsulfoxide (DMSO), dimethylacetamide, N-methyl pyrrolidone (NMP), acetic acid, trifluoroacetic acid, ethyl acetate, acetonitrile, trifluoroacetic anhydride, 1,1,1-trifluoroacetone, maleic acid, hexafluoroacetone.

In general, when producing fibers using electrospinning techniques, the base material that is used can be the monomer of the polymer fiber to be formed. In some embodiments it is desirable to use monomers to produce finer filaments. In other embodiments, it is desirable to include partial fibers to add material strength to the matrix and to provide additional sites for incorporating substances.

In addition to the multiple equipment variations and modifications that can be made to obtain desired results, similarly the electrospun solution can be varied to obtain different results. For instance, any solvent or liquid in which the material is dissolved, suspended, or otherwise combined without deleterious effect on the process or the safe use of the matrix can be used. Materials or the compounds that form materials can be mixed with other molecules, monomers or polymers to obtain the desired results. In some embodiments, polymers are added to modify the viscosity of the solution. In still a further variation, when multiple reservoirs are used, the ingredients in those reservoirs are electrosprayed separately or joined at the nozzle so that the ingredients in the various reservoirs can react with each other simultaneously with the streaming of the solution into the electric field. Also, when multiple reservoirs are used, the different ingredients in different reservoirs can be phased in temporally during the processing period. These ingredients may include other substances.

Embodiments involving alterations to the electrospun materials themselves are within the scope of the present invention. Some materials can be directly altered, for example, by altering their carbohydrate profile. Also, other materials can be attached to the matrix materials before, during or after electrospinning using known techniques such as chemical cross-linking or through specific binding. Further, the temperature and other physical properties of the process can be modified to obtain different results. The matrix may be compressed or stretched to produce novel material properties.

Electrospinning using multiple jets of different polymer solutions and/or the same solutions with different types and amounts of substances (e.g., growth factors) can be used to prepare libraries of biomaterials for rapid screening. Such libraries are desired by those in the pharmaceutical, advanced materials and catalyst industries using combinatorial synthesis techniques for the rapid preparation of large numbers (e.g., libraries) of compounds that can be screened. For example, the minimum amount of growth factor to be released and the optimal release rate from a fibrous polymer scaffold to promote the differentiation of a certain type of cell can be investigated using the compositions and methods of the present invention. Other variables include fiber diameter and fiber composition. Electrospinning permits access to an array of samples on which cells can be cultured in parallel and studied to determine selected compositions which serve as promising cell growth substrates.

One of ordinary skill in the art recognizes that changes in the concentration of materials or substances in the solutions requires modification of the specific voltages to obtain the formation and streaming of droplets from the tip of a pipette.

The electrospinning process can be manipulated to meet the specific requirements for any given application of the electrospun compositions made with these methods. In one embodiment, the micropipettes can be mounted on a frame that moves in the x, y and z planes with respect to the grounded substrate. The micropipettes can be mounted around a grounded substrate, for instance a tubular mandrel. In this way, the materials or molecules that form materials streamed from the micropipettes can be specifically aimed or patterned. Although the micropipettes can be moved manually, the frame onto which the micropipettes are mounted can be controlled by a microprocessor and a motor that allow the pattern of streaming collagen to be predetermined by a person making a specific matrix. Such microprocessors and motors are known to one of ordinary skill in the art. For instance, matrix fibers or droplets can be oriented in a specific direction, they can be layered, or they can be programmed to be completely random and not oriented.

In the electrospinning process, a material stream or streams can branch out to form fibers. The degree of branching can be varied by many factors including, but not limited to, voltage, ground geometry, distance from micropipette tip to the substrate, diameter of micropipette tip, and concentration of materials or compounds that will form the electrospun materials. As noted, not all reaction conditions and polymers may produce a true multifilament, under some conditions a single continuous filament is produced. Materials and various combinations can also be delivered to the electric field of the system by injecting the materials into the field from a device that will cause them to aerosol. This process can be varied by many factors including, but not limited to, voltage (for example ranging from about 0 to 30,000 volts), distance from micropipette tip to the substrate (for example from 0-40 cm), the relative position of the micropipette tip and target (i.e. above, below, aside etc.), and the diameter of micropipette tip (approximately 0-2 mm).

In some embodiments, the electroprocessed GelMA compositions include additional electroprocessed materials. For example, other electroprocessed materials can include natural materials, synthetic materials, or combinations thereof. Examples include, but are not limited, to amino acids, peptides, denatured peptides such as gelatin from denatured collagen, polypeptides, proteins, carbohydrates, lipids, nucleic acids, glycoproteins, minerals, lipoproteins, glycolipids, glycosaminoglycans, and proteoglycans.

In some embodiments, the composition of the present invention includes additional electroprocessed materials. Other electroprocessed materials can include natural materials, synthetic materials, or combinations thereof. Some examples of natural materials include, but are not limited to, amino acids, peptides, denatured peptides such as gelatin from denatured collagen, polypeptides, proteins, carbohydrates, lipids, nucleic acids, glycoproteins, lipoproteins, glycolipids, glycosaminoglycans, and proteoglycans. Some synthetic matrix materials for electroprocessing with collagen include, but are not limited to, polymers such as poly(lactic acid) (PLA), polyglycolic acid (PGA), copolymers of PLA and PGA, polycaprolactone, poly(ethylene-co-vinyl acetate), (EVOH), poly(vinyl acetate) (PVA), polyethylene glycol (PEG) and poly(ethylene oxide) (PEO).

### Kits

The present invention also includes kits comprising components useful within the methods of the invention and instructional material that describes, for instance, the method of using the scaffolds. The kit may comprise components and materials useful for performing the methods of the invention. For instance, the kit may comprise GelMA and Bio-IL and spinning solutions. In certain embodiments, the kit may comprise preformed scaffolds. In other embodiments, the kit further comprises cell cultures and surgical instruments.

In one embodiment, the kit is for cardiac tissue regeneration. For example, the kit may comprise scaffolds having preset sizes, such as small, medium, large, and extra-large, wherein an operator may select an appropriate kit having an appropriately sized scaffold. The kit may further comprise bandages, antibiotics, or other drugs to enhance tissue regeneration.

In some embodiments, the kit may further comprise scaffolds placed in a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea, and combinations thereof. In one embodiment, the preservative is a combination of about 0.5% to 2.0% benzyl alcohol and 0.05% to 0.5% sorbic acid.

In certain embodiments, the kit comprises instructional material. Instructional material may include a publication, a recording, a diagram, or any other medium of expression which can be used to communicate the usefulness of the device or implant kit described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention and are not to be construed as limiting in any way the remainder of the disclosure.

### Example 1: Engineering a Naturally-Derived Adhesive and Conductive Cardiopatch

Myocardial infarction (MI) leads to a multi-phase reparative process at the site of damaged heart that ultimately results in the formation of non-conductive fibrous scar tissue. Despite the widespread use of electroconductive biomaterials to increase the physiological relevance of bioengineered cardiac tissues in vitro, there are still several limitations associated with engineering biocompatible scaffolds with appropriate mechanical properties and electroconductivity for cardiac tissue regeneration. Here, a highly adhesive fibrous scaffolds engineered by electrospinning of gelatin methacryloyl (GelMA) followed by the conjugation of a choline-based bio-ionic liquid (Bio-IL) to develop conductive and adhesive cardiopatches is introduced. These GelMA/Bio-IL adhesive patches were optimized to exhibit mechanical and conductive properties similar to the native myocardium. Furthermore, the engineered patches strongly adhered to murine myocardium due to the formation of ionic bonding between the Bio-IL and native tissue, eliminating the need for suturing. Co-cultures of primary cardiomyocytes and cardiac fibroblasts grown on GelMA/Bio-IL patches exhibited comparatively better contractile profiles compared to pristine GelMA controls, as demonstrated by over-expression of the gap junction protein connexin 43. These cardiopatches could be used to provide mechanical support and restore electromechnical coupling at the site of MI to minimize cardiac remodeling and preserve normal cardiac function.

The materials and methods employed in these experiments are now described.

### Cardiopatch Fabrication

Porcine GelMA was synthesized as described previously (J. W. Nichol et al., 2010, Biomaterials, 31(21):5536-44). A prepolymer solution was then prepared by mixing 10, 12.5, and 15% (w/v) of GelMA in hexafluoroisopropanol (HFIP) (Sigma-Aldrich), and placed in a syringe with a 27G needle. The prepolymer solution was then pumped out of the syringe at a rate of 1 mL/h. A high voltage power source (Glassman High Voltage, Inc., Series EH) was attached to the needle of the syringe, and to a metal collector that the GelMA polymer was drawn to, creating a fibrous mat. Fibrous scaffolds were then removed from the collector plate and placed in a vacuum to remove any remaining solvent. Scaffolds were then placed in a solution bath containing 1.25% (w/v) photoinitiator Irgacure 2959 (Sigma-Aldrich) in ethanol. Bio-IL was also synthesized using the previously discussed methodology (I. Noshadi et al., 2017, Sci Rep 7(1):4345). Four concentrations of Bio-IL in water were prepared including 0, 33, 66, and 100% (v/v). Scaffolds were placed in a refrigerator to prevent the dissolving of GelMA fibers in Bio-IL/water solution. A volume of 1 mL Bio-IL was then placed on the surface of GelMA fibrous scaffolds and immediately crosslinked using UV irradiation for 300 seconds on each side of the scaffold.

### H NMR Characterization

H NMR analysis was performed using a Varian Inova-500 NMR spectrometer. H NMR spectra were obtained for a choline-based Bio-IL prepolymer, GelMA, prepolymer, GelMA fibers after UV photocrosslinking, and Bio-IL/GelMA cardiopatches. Methacrylated groups were identified due to the presence of peak values at δ=5.3, and 5.7 ppm. The decreasing rate for the C═C double bond signals

$$( - \frac{\partial ( C = C )}{\partial t} )$$

in methacrylate group of GelMA was associated with the extent of crosslinking of cardiopatches, as well as conjugation of GelMA to Bio-IL. This area decrease was calculated using the following equation:

$$Decay of methacrylate group ( \% ) = ( \frac{P {A}_{b} - {PA}_{a}}{P {A}_{b}} ) \times 100 \%$$

where PA_{b}, and PAₐ represent the peak areas of methacrylated groups before and after photocrosslinking, respectively. Accordingly, PA_{b}−PAₐ corresponds to the concentration of methacrylated groups consumed in the photo-crosslinking process. ACD/Spectrus NMR analysis software were used to integrate the area under the peaks and all the data was analyzed with respect to phenyl group peaks at δ=6.5-7.5 ppm.

### Scanning Electron Microscopy (SEM) Analysis

The diameter and morphology of the electrospun nanofibrous sheets were examined by SEM; Hitachi S-4800, Japan. Prior to imaging, the samples were fixed in 2% osmium tetroxide (OsO₄, Fisher Scientific). The scaffolds were then washed three times with DPBS each for 5 min, followed by dehydration in graded ethanol series (i.e., 30, 50, 70, 95, and 100% v/v) each for 10 min. Next, samples were dried at critical point with a Tousimis critical point dryer. After drying, the scaffolds were sputter coated with gold/palladium (6 μm). The obtained images were processed by ImageJ software to determine the average fiber diameter sizes (50 arbitrary fibers per each group).

### In Vitro Evaluation of Conductivity

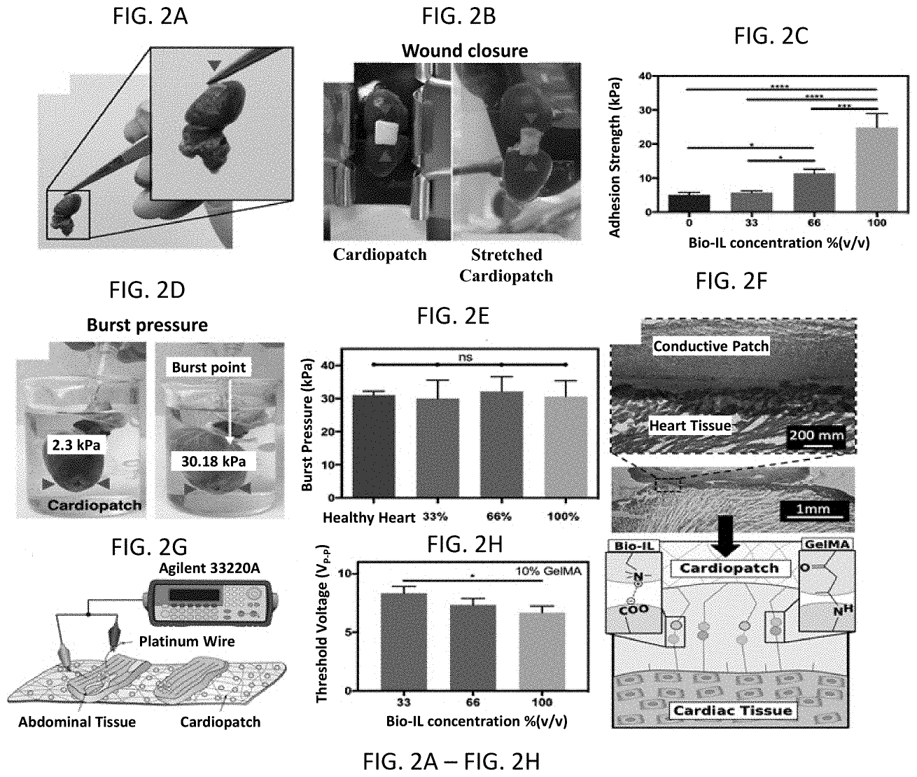

Cardiopatches were photocrosslinked with UV irradiation for 300 seconds on each side and allowed to dry for 24 h. Once dried, conductivity analysis was performed using a two-probe electrical station connected to a Semiconductor Parameter analyzer, as previously described (FIG. 2A) (Noshadi, I. et al., 2017, Sci Rep-UK, 7(1):4345). The results were then analyzed to determine the electrical conductivity of cardiopatches. Cardiopatches were also examined for conductivity following degradation in DPBS at 37° C. for a period of 0, 2, and 4 d. Samples were removed from DPBS and allowed to dry for 24 h. Electrical conductivity was then measured using the same protocol to measure electrical conductivity in samples that had not degraded. The conductivity of cardiopatches was also determined using methods previously described (Noshadi, I. et al., 2017, Sci Rep-UK, 7(1):4345). Cardiopatches were also examined for conductivity under stretched conditions. Briefly, cardiopatches were fabricated using the same method as above, but were dried for only 2 h to prevent brittleness. The trace amount of moisture led to increased conductivity readings, however, allowed samples to be mechanically stretched without breaking. Samples were stretched at a strain of 0, 20, and 40% and conductivity was measured using the same method as above. At least 5 samples were tested for each condition.

### Swelling Ratio Measurements

Cardiopatches of varying GelMA and Bio-IL concentrations were synthesized as described previously and cut into small pieces. The small pieces were then lyophilized, weighed, and placed in DPBS at 37° C. At prearranged time points (4, 8, 24 h), samples were removed and weighed again after immersion. The swelling of the samples was calculated as the ratio of the swelled mass to the mass of the lyophilized sample.

### In Vitro Degradation Test

Cardiopatches were synthesized as previously described, cut into small square sections, and lyophilized overnight. Samples were weighed and placed in 1.5 mL tubes of 1 mL DPBS with 5.0 U/mL collagenase type II, and incubated at 37° C. for up to 72 h. The collagenase solution was refreshed every 24 h. At prearranged points (after 6, 12, 24, 48, and 72 h), the collagenase solution was removed, and samples were lyophilized for 24 h and weighed. The percentage of degradation (D %) of the cardiac patches was calculated using the below equation:

$$D \% = \frac{{W}_{i} - {W}_{t}}{{W}_{i}} \times 100 \%$$

where Wₜ is the initial dry weight of the patch, and Wₜ is the dry weight after time t.

### Mechanical Testing

Tensile test was performed on cardiac patches using an Instron 5944 mechanical tester using method previously described (I. Noshadi et al., 2017, Sci Rep-UK, 7(1):4345). At least 5 samples were tested for each condition.

### Wound Closure Test

Wound closure tests were performed using a modified ASTM F2458-05 to determine the adhesive strength based on the previously explained procedures (Annabi, N. et al., 2017, Sci Transl Med, 9(410); Annabi, N. et al., 2017, Biomaterials, 139:229-243; Chandrasekharan, A. et al., 2019, Journal of Polymer Science Part A: Polymer Chemistry, 57(4):522-530). Porcine skin and rat myocardium wet tissues were used as substrates. Briefly, samples of the biological substrate were cut into 40×20 mm pieces with a thickness of approximately 5 mm. The substrate was immersed in DPBS to prevent drying. Tissue samples were then glued with cyanoacrylate adhesive onto glass slides. Two sections of the substrate were then placed against each other, and a cardiopatch was photocrosslinked for 300 seconds over the tissues to glue them together. An Instron mechanical tester was used to measure the maximum adhesive strength at the point of patch failure.

### Burst Pressure Test

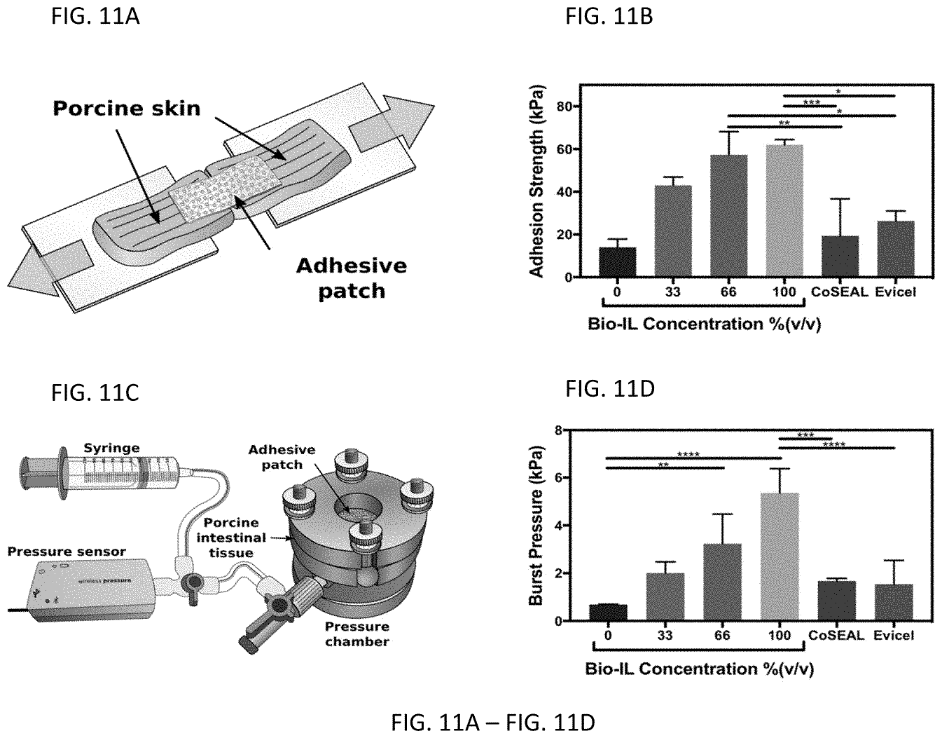

Burst pressure adhesion test was performed using a modified ASTM F2392-04 for determining the sealing strength of a biomaterial. Collagen sheets were used as substrates. First, the collagen sheet was soaked in DPBS for 1 h and placed between two Teflon plates and placed into a custom-designed burst pressure apparatus. A 3 mm defect was then created into the substrate using a surgical blade. Cardiopatches were then fabricated and photocrosslinked on the defect site, and air pressure was increased until patch failure (FIG. 11C).

A modified ex vivo burst pressure test was conducted using cardiopatches photocrosslinked on freshly explanted rat hearts according to previously published reports (Li, J. et al., 2017, Science, 357(6349):378-381). Briefly, an air tube was fed through the top of excised rat hearts into the LV, and a defect was created on the myocardial wall of the LV using a surgical blade (2 mm). Cardiopatches were photocrosslinked onto the defect site. Rat hearts were then placed in a beaker containing water and air pressure was increased in the LV until patch failure.

### Ex Vivo Evaluation of Electrical Conductivity

Adult female Wistar rats were provided by the Institutional Animal Care and Use Committee (IACUC) at Northeastern University (Boston, Mass., USA). All experiments were performed in accordance with relevant guidelines and regulations. Immediately after euthanasia, the rectus abdominus tissue was removed from Wistar rats and placed in DPBS. The rectus abdominus was cut into small square pieces and placed adjacently with a 3 mm gap on cardiopatches with varying Bio-IL concentration. 50 ms square pulses of direct current were applied to the tissue using an Agilent wave generator (Agilent 33220A). The electrical stimulation was applied to one piece of abdominal tissue using short platinum wires with 0.25 mm diameter and 99.9% trace metal basis, bought from Sigma-Aldrich (MO, USA). The threshold was measured by increasing voltage applied to one section of abdominal tissue and observing the lowest voltage at which the neighboring section of tissue contracted.

### Surface Seeding of Primary CMs and CFs on GelMA/Bio-IL Cardiopatches

A thin layer of 10% (w/v) GelMA was electrospun onto 0.8×0.8 cm glass slides, coated with 3-(trimethoxysilyl) propyl methacrylate (TMSPMA). The glass slides were then soaked in 1.25% (w/v) Irgacure 2959 solution for 1 h, and kept at −80° C. for 1 min. A conductive layer was then formed on top of the electrospun GelMA by pipetting a 50-μ1 drop of Bio-IL at different concentrations (i.e., 0%, 33%, 66%, and 100% (v/v)), followed by UV-initiated photocrosslinking for 5 min. The samples were incubated overnight in Dulbecco's Modified Eagle Medium (DMEM) supplemented with 10% Nu-Serum growth supplement, and 1% penicillin/streptomycin. Primary CMs and CFs were isolated from neonatal rat hearts as described previously (Noshadi, I. et al., 2017, Sci Rep, 7(1):4345). Co-cultures of CMs/CFs were then seeded at a ratio of 2:1 on top of the scaffolds at a density of 2×10⁵ cells/cm² and maintained at 37° C., in a 5% CO₂ humidified atmosphere for up to 7 days. Cell viability, and metabolic activity were determined at days 1, 4, and 7 post-seeding as described in the previous publication (Noshadi, I. et al., 2017, Sci Rep, 7(1):4345). IFS against cardiac markers SAA and Cxs43 was carried out as described previously (Noshadi, I. et al., 2017, Sci Rep, 7(1):4345).

### In Vivo Evaluation of Biosafety and Cardioprotective Potential of Electrospun Scaffolds

All experiments were performed according to the protocol approved by the IACUC. Experimental MI was induced via permanent ligation of the LAD as described previously (Kolk, M. V. et al., 2009, J Vis Exp (32)). Immediately after induction of MI, the scaffolds were delivered to the surface of the left ventricle, distal to the site of MI, and photocrosslinked for 300 seconds using UV light. To remove any unreacted Bio-IL, saline was pipetted to the surface of cardiopatches and excess liquid was collected using a gauze pad. Animals were divided into three groups: sham (control), pristine GelMA patches (i.e., 10% (w/v) GelMA), and GelMA/Bio-IL patches (i.e., 33% (v/v) Bio-IL and 10% (w/v) GelMA). There were 3 animals per group. Following administration of the treatments, the animals were allowed to recover after anatomical wound closure and followed for a period of 3 weeks. After this period, the animals were euthanized, and the hearts were removed and processed for histological evaluation and IFS as described previously (Noshadi, I. et al., 2017, Sci Rep, 7(1):4345).

The results of the experiments are now described

### Physicochemical Characterization of GelMA/Bio-IL Cardiopatches

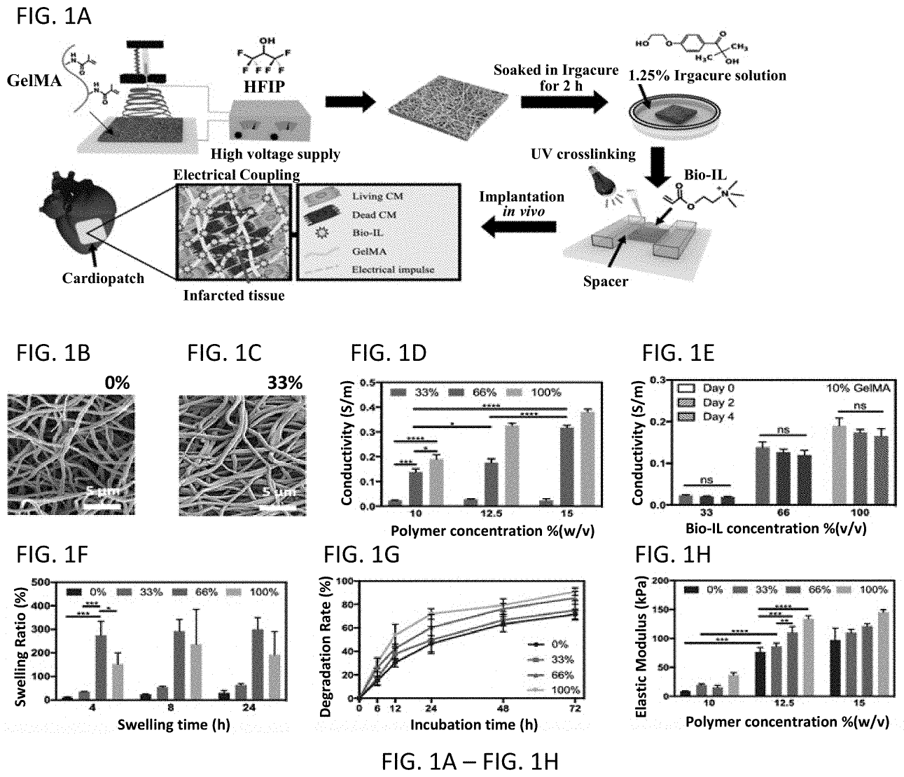

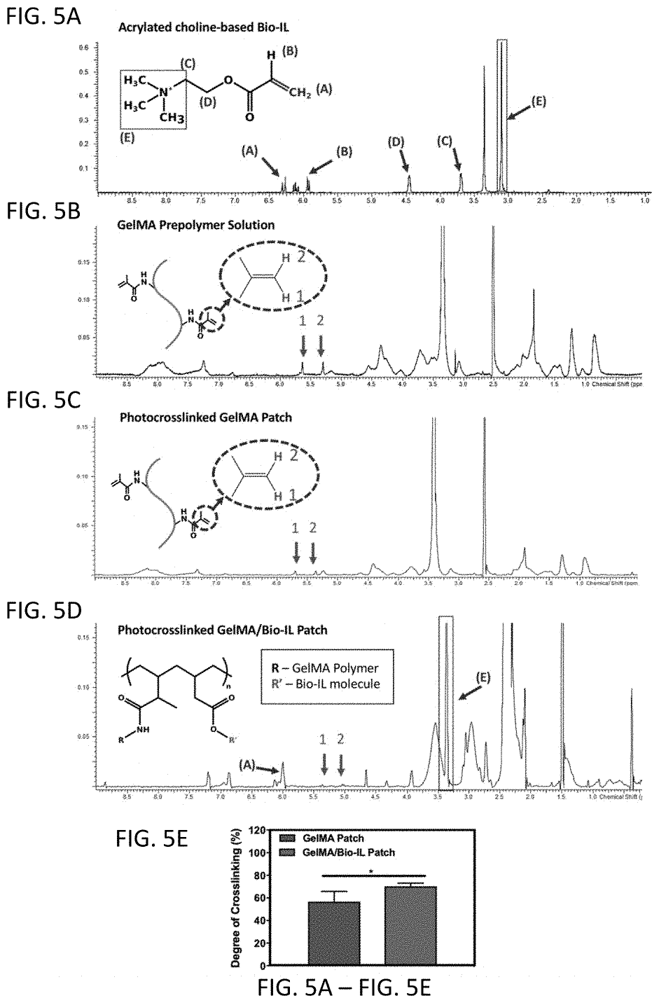

Fibrous patches were prepared by first electrospinning different concentrations of the GelMA precursor mixed with 1,1,1,3,3,3-hexafluoro-2-propanol (HFIP), onto a static metal collector. Electrospun patches were then incubated in 1.25% (w/v) Irgacure 2959 in ethanol, followed by direct addition of various concentrations of Bio-IL and crosslinking via exposure to UV light for 5 min (FIG. 1A). Chemical conjugation of Bio-IL to GelMA was first confirmed via proton nuclear magnetic resonance (H NMR) as described previously (Noshadi, I. et al., 2017, Sci Rep, 7(1):4345). Briefly, H NMR spectra were obtained for Bio-IL, GelMA prepolymer, photocrosslinked GelMA patches, and photocrosslinked GelMA/Bio-IL patches (FIG. 5A-FIG. 5D). Then the degree of consumption of C═C double bonds in methacryloyl groups during free radical polymerization was determined, which occurred both due to the crosslinking of GelMA and the conjugation of Bio-IL to GelMA. Results showed that 70.2±7.8% of methacryloyl groups were consumed after photocrosslinking of GelMA/Bio-IL patch, which was significantly higher than those calculated for pure photocrosslinked GelMA patch (56.7±9.1%) (FIG. 5E). The larger rate of decay of the C═C double bonds in GelMA/Bio-IL patches represents the chemical conjugation of acrylate groups in Bio-IL to methacryloyl groups in GelMA (FIG. 5D).

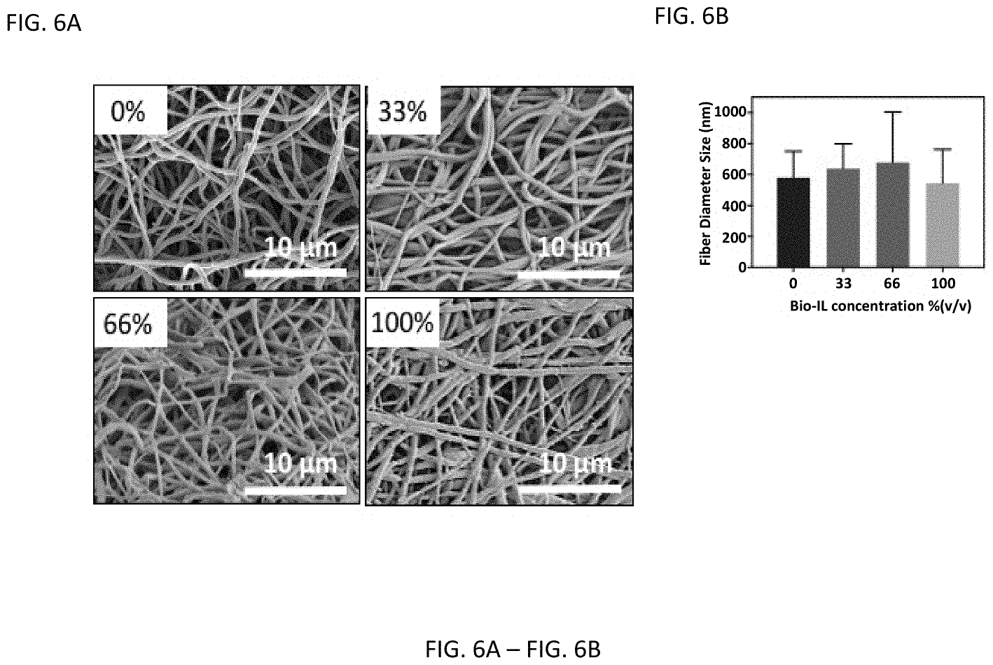

The native cardiac ECM is comprised of several structural fibrillar proteins such as collagen and elastin, which range from 10 to several hundred nanometers in diameter (Dvir, T. et al., 2011, Nat Nanotechnol, 6(1):13-22). The formation of biomimetic fibrous structures plays an important role in the physical characteristics of TE scaffolds, such as their mechanical strength, porosity, and surface area/volume ratio (Zhao, G. et al., 2015, Adv Func Mat, 25(36):5726-5738). Hence, the aim was to characterize the fiber topology of GelMA/Bio-IL cardiopatches synthesized with varying concentrations of Bio-IL via scanning electron microscopy (SEM) (FIG. 1B-FIG. 1C, FIG. 6). SEM images showed no significant differences in the fiber diameter when different concentrations of Bio-IL were used. The fiber diameter was in the range of 544.0±218.6 nm to 676.7±326.2 nm for the engineered cardiopatches. These results demonstrated that Bio-IL conjugation does not influence fiber diameter, enabling the ability to produce scaffolds with tunable conductivity without varying the microstructures.

### Electroconductive Properties of GelMA/Bio-IL Cardiopatches

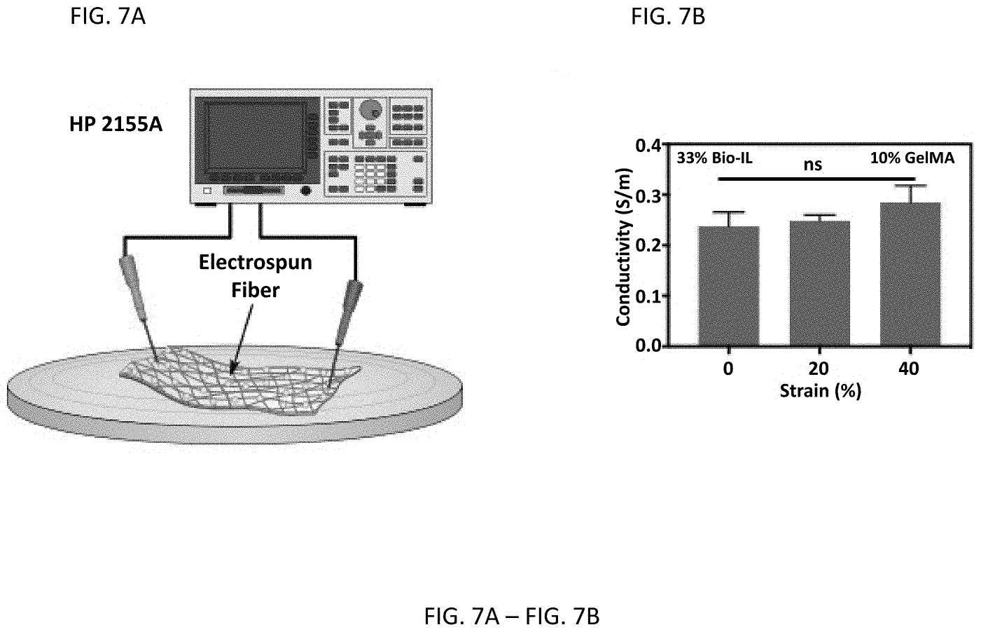

The conductivity of the engineered patches was analyzed as previously described (FIG. 7A) (Noshadi, I. et al., 2017, Sci Rep, 7(1):4345). Results showed that the conductivity of the scaffolds could be tuned by varying the concentrations of both GelMA and Bio-IL (FIG. 1D). For instance, the electrical conductivity of scaffolds fabricated with 10% (w/v) GelMA increased from 0.23×10⁻¹±0.02×10⁻¹ S/m to 1.38×10⁻¹±0.12×10⁻¹ S/m, and 1.90×10⁻¹±0.18×10⁻¹ S/m, when the Bio-IL concentration was increased from 33% to 66%, and 100% (v/v), respectively. As expected, GelMA patches fabricated with 0% (v/v) Bio-IL did not demonstrate any conductivity, as GelMA itself is not inherently conductive. On the other hand, patches fabricated with 66% (v/v) Bio-IL exhibited an increase in electrical conductivity from 1.38×10⁻¹±0.13×10⁻¹ S/m to 1.77×10⁻¹±0.15×10⁻¹ S/m, and 3.18×10⁻¹±0.01×10⁻¹ S/m when the GelMA concentration increased from 10% to 12.5%, and 15% (w/v), respectively. Though GelMA itself is not inherently conductive, this increase in conductivity may be attributed to more functional groups available in the GelMA prepolymer at higher concentration that can react with Bio-IL. Furthermore, these values are within the range of the electrical conductivity of the native myocardium, which has been shown to be between 1.6×10⁻¹ S/m (longitudinally) and 0.05×10⁻¹ S/m (transversally) (Qazi, T. H. et al., 2014, Acta Biomat, 10(6):2434-45).

The conductivity of the scaffolds was also characterized after 0, 2, and 4 days of incubation in Dulbecco's phosphate buffered saline (DPBS) at 37° C. to determine the effect of scaffold degradation on electrical conductivity. These results showed that the conductivity of GelMA/Bio-IL cardiopatches exhibited no statistically significant differences after up to 4 days of incubation for all conditions tested (FIG. 1E). Furthermore, the conductivity of GelMA/Bio-IL cardiopatches under mechanically strained conditions were evaluated to determine the effect of scaffold deformation on electrical conductivity. For this, the scaffolds were first dried for 2 h to retain trace amounts of moisture and prevent stiffening. The presence of moisture led to increased conductivity readings as compared to dried samples (FIG. 1D), however, allowed samples to be mechanically stretched without breaking. The samples were then stretched at a strain rate of 20% and 40%, and electrical conductivity was measured in the stretched state as described before (Noshadi, I. et al., 2017, Sci Rep, 7(1):4345). Results showed that there were no statistically significant differences in conductivity of the GelMA/Bio-IL cardiopatches when stretched up to 40% strain, compared to static conditions (FIG. 7B). These results demonstrated that the conductivity of the scaffolds remained unaffected following degradation or stretching, which is critical to maintain a consistent supportive microenvironment for the excitable phenotypes that comprise the contractile myocardium.

Here, it was demonstrated that systematic variations in the formulation of GelMA/Bio-IL cardiopatches yielded scaffolds with a wide range of physicochemical properties. Bio-IL conjugation provided GelMA-based scaffolds with highly tunable electrical conductivity (FIG. 1D) without affecting the fiber diameter of the scaffolds (FIG. 1B-FIG. 1C). Due to the continuous movement of heart due to beating, it was aimed to investigate the conductive properties of these cardiopatches while under mechanical tension, and after up to four days of degradation in vitro. Results showed that the conductive properties of the scaffolds were not affected by the degradation (FIG. 1E) or deformation (FIG. 7B) of the conductive cardiopatches. Thus, there should not be a significant drop in the conductive properties of the patches while adhered to the heart due to the contraction or expansion of cardiac tissue. Cardiopatches, therefore, should support the electrical pathways of the myocardium by aiding in the propagation of electrical signals throughout all phases of cardiac cycle. This is in contrast to other composite hydrogels fabricated with conductive nanoparticles where the inter-particle distance plays a key role in the conductive properties of the scaffold (Thoniyot, P. et al., 2015, Adv Sci 2, 1(2):1400010).

### In Vitro Swellability and Degradation Rates of GelMA/Bio-IL Cardiopatches

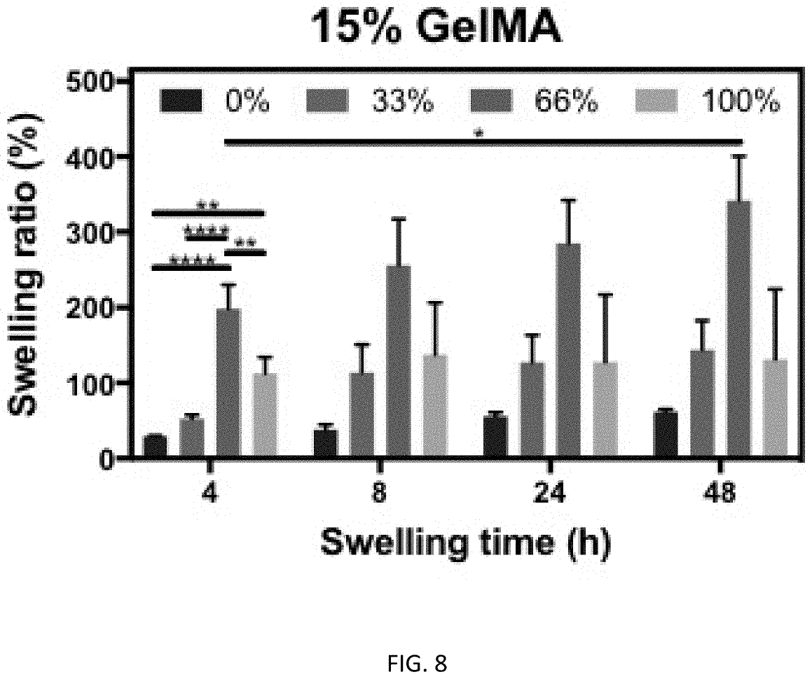

Excess water intake could potentially compromise the mechanical and conductive properties of TE scaffolds. Hence, it was aimed to evaluate the water uptake capacity of GelMA/Bio-IL cardiopatches. Results showed that scaffolds fabricated with 10% (w/v) GelMA and varying Bio-IL concentrations swelled rapidly after 4 h of incubation, with no significant increases in water uptake after 8 and 24 h for all Bio-IL concentrations (FIG. 1F). Results also showed that scaffolds fabricated with 66% and 100% (v/v) Bio-IL underwent significantly higher swelling when compared to scaffolds fabricated with a lower Bio-IL concentration. This behavior could be explained in part due to the presence of hydroxyl (—OH) and amine (—NH₂) hydrophilic groups in the Bio-IL structure, which enhances the swelling ratio. There was no statistically significant difference in the swelling ratio of cardiopatches fabricated with 66% and 100% Bio-IL after 8 h. However, a decrease in swelling ratio from 66% to 100% (v/v) Bio-IL concentration after 4 h was observed. This can be explained, in part, due to a higher percentage of Bio-IL that is not conjugated to the GelMA polymer network and is washed out in the first 4 h while submerged in DPBS. Furthermore, scaffolds fabricated with 15% (w/v) GelMA showed a similar trend, with higher swelling ratios obtained at higher concentrations of Bio-IL (FIG. 8).

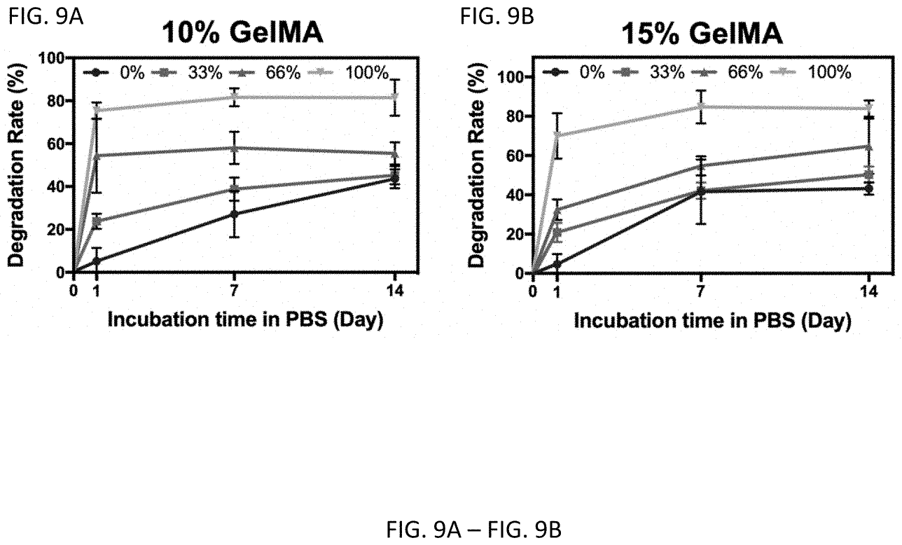

Following implantation, TE scaffolds should biodegrade into nontoxic byproducts to allow the growth of new autologous tissue (Martins, A. M. et al., 2014, Biomacromolecules, 15(2):635-43). Thus, it was aimed to characterize the in vitro enzymatic degradation profile of GelMA/Bio-IL cardiopatches. Briefly, scaffolds were lyophilized and weighed, followed by incubation in DPBS and 5.0 U/mL of collagenase type II solution at 37° C. for up to 72 h. At the end of this period, the samples were lyophilized and re-weighed to determine the changes in dry mass after degradation. The collagenase solution was replaced daily. Results showed that the degradation rate increased concomitantly when the Bio-IL concentration was increased for cardiopatches containing 10% (w/v) GelMA (FIG. 1G). For example, results show that following 24 h of incubation in collagenase type II solution, cardiopatches demonstrated a degradation rate of 49.65±11.60% and 71.90±4.55% for scaffolds fabricated with 33% (v/v) and 100% (v/v) Bio-IL, respectively. In addition, the in vitro degradation profile of the cardiopatches incubated in DPBS solution were also evaluated. Results showed that after 1 day of incubation, scaffolds fabricated using 10% GelMA and 33% (v/v) Bio-IL exhibited degradation rates corresponding to 25.75%±3.57% (FIG. 9). At 14 days following incubation, cardiopatches containing 10% (w/v) GelMA and 33% (v/v) Bio-IL exhibited 45.32±4.27% degradation (FIG. 9). Moreover, the degradation rate increased concomitantly when the Bio-IL concentration was increased for cardiopatches containing both 10% and 15% (w/v) GelMA. In addition, no statistical differences were observed between the degradation rate of the patches fabricated with 10% and 15% (w/v) GelMA after 1 and 14 days. Both degradation studies performed in DPBS as well as in a collagenase solution demonstrated that more rapid degradation occurred in cardiopatches fabricated with a higher concentration of Bio-IL. This trend could be attributed in part to higher amounts of unconjugated Bio-IL that were washed out of the hydrogel network.

### Mechanical Characterization of GelMA/Bio-IL Cardiopatches

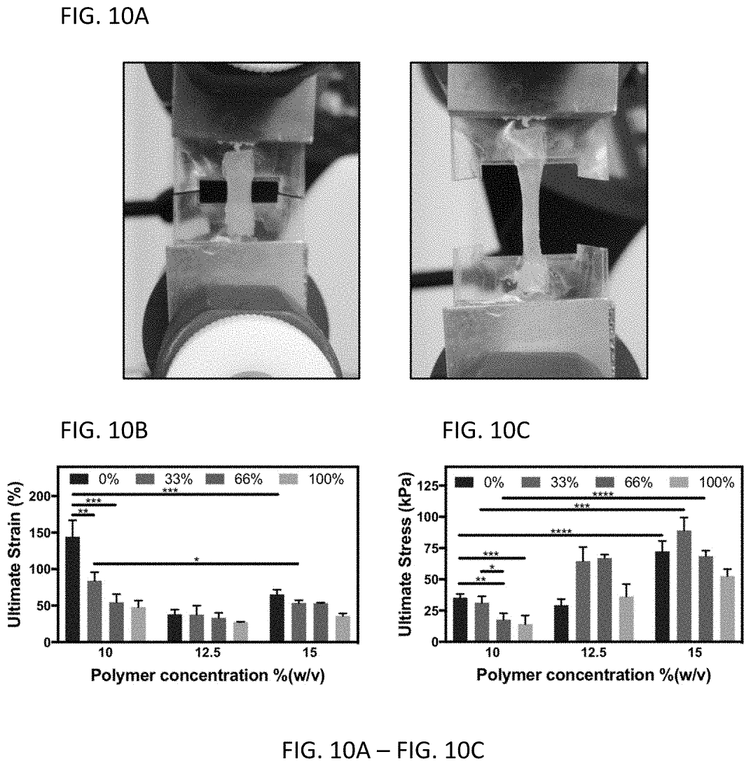

Scaffolds used for cardiac TE should possess similar mechanical properties to the native myocardium to prevent mechanical mismatches that could impair contractile function of native heart (Radhakrishnan, J. et al., 2014, Biotechnol Adv, 32(2):449-461; Liau, B. et al., 2012, Regen Med, 7(2):187-206). Thus, the mechanical properties of scaffolds fabricated was evaluated using varying concentrations of GelMA and Bio-IL (FIG. 1H and FIG. 10A). The results showed that the engineered patches exhibited highly tunable elastic moduli (i.e., 8.76±0.42 kPa to 145.50±4.10 kPa), which was in the range of the stiffness reported for the native myocardium (˜20-100 kPa) (Ebrahimi, A. P. et al., 2009, J Vasc Intery Neurol, 2(2):155-162; Fioretta E. S. et al., 2012, J Biomech, 45(5):736-744). Also, the elastic moduli increased concomitantly with increasing GelMA concentrations (FIG. 1H). For instance, the elastic moduli of scaffolds fabricated with 33% (v/v) Bio-IL increased from 19.67±1.70 kPa to 86.23±5.61 kPa, and 110.00±5.56 kPa when the concentration of GelMA increased from 10% to 12.5%, and 15% (w/v), respectively (FIG. 1H). Results also showed that the elastic moduli of the scaffolds could also be increased by increasing the concentration of Bio-IL. For instance, the elastic moduli of scaffolds fabricated with 12.5% (w/v) GelMA increased from 86.23±5.61 kPa to 110.45±9.97 kPa, and 134.06±5.06 kPa when the concentration of Bio-IL was increased from 33% to 66%, and 100% (v/v), respectively (FIG. 1H). This increase in mechanical properties of the patches may be due to the electrostatic interactions between the positively charged groups in Bio-IL and the negatively charged functional groups present in the GelMA polymer. Ionic interactions, such as these, have previously been shown to increase mechanical strength in hydrogels (Wang, W. et al., 2017, Prog Polym Sci, 71:1-25). In addition, there might be chemical bonding between the Bio-IL and GelMA prepolymer through photopolymerization, which can also increase the mechanical properties of the patches. Furthermore, the ultimate strain and ultimate stress of the scaffolds were also shown to vary by changing the concentrations of both Bio-IL and GelMA (FIG. 10). For instance, the ultimate strain of scaffolds with 10% (w/v) GelMA decreased from 84.2±11.46 kPa to 47.9±8.91 kPa when the concentration of Bio-IL was increased from 33% to 100% (v/v), respectively (FIG. 10B). Moreover, the ultimate stress of scaffolds fabricated with 33% (v/v) Bio-IL increased from 31.31±5.18 kPa to 64.59±11.19 kPa, and 89.03±10.41 kPa when the concentration of GelMA was increased from 10% to 12.5%, and 15% (w/v), respectively (FIG. 10C). Taken together, these results demonstrated the remarkable mechanical tunability of GelMA/Bio-IL cardiopatches, which is highly advantageous for the engineering of electroconductive scaffolds for a variety of TE and biomedical applications.

The engineered patches did not exhibit any significant increase in their water uptake capacity after 4 h, and up to 24 h of incubation in DPBS (FIG. 1F). In addition, the results also showed that the elastic moduli of GelMA/Bio-IL cardiopatches could be tuned by varying the concentration of GelMA and Bio-IL, and that the mechanical properties of the scaffolds were within the range of the native human myocardium (FIG. 1H). These characteristics highlight the remarkable potential of GelMA/Bio-IL cardiopatches to be used as cardio-supportive devices, owing to their high electrical conductivity and biocompatibility, controlled swellability and degradability, as well as their biomimetic fibrillar topology and mechanical properties.

### Adhesive Properties of GelMA/Bio-IL Cardiopatches to Physiological Tissues

Biomaterials with strong adhesive properties to wet tissues have emerged as promising strategies for sutureless wound closure following surgical procedures (Feng, G. et al., 2016, Macromol Biosci, 16(7):1072-1082). In this regard, in the previous studies, it was demonstrated that GelMA-based hydrogels possess high adhesive strength to various physiological tissues, while also exhibiting superior mechanical performance when compared with commercially available tissue adhesives (Assmann, A. et al., 2017, Biomaterials, 140:115-127; Annabi, N. et al., 2017, Biomaterials, 139:229-243). Here, it was aimed to evaluate the adhesive strength of GelMA/Bio-IL cardiopatches to the native myocardium to determine their potential for sutureless application following MI. The standard wound closure and burst pressure tests from the American Society for Testing and Materials (ASTM) was used, as well as ex vivo experiments using murine cardiac tissue to evaluate the adhesive properties of the engineered cardiopatches. First, wound closure experiments were carried out to evaluate the adhesive strength of the scaffolds to porcine skin (FIG. 11A-FIG. 11B), and murine left ventricular myocardium (FIG. 2A-FIG. 2C). Wound closure tests showed that the adhesiveness of GelMA/Bio-IL cardiopatches to porcine skin increased up to 61.97±2.50 kPa by increasing the concentration of Bio-IL (FIG. 11B). Furthermore, the adhesive strength of scaffolds synthesized using 66% and 100% (v/v) Bio-IL was shown to be significantly higher than the commercial surgical sealants such Coseal™ (19.4±17.3 kPa) and Evicel® (26.3±4.7 kPa) (FIG. 11B). In addition, visual inspection revealed that scaffolds photocrosslinked on the surface of the tissue also adhered strongly to the native murine myocardium (FIG. 2A). Similarly, wound closure tests on murine myocardium (FIG. 2B) revealed that the adhesiveness of GelMA/Bio-IL cardiopatches increased from 5.1±0.4 to 24.89±2.34 kPa as the Bio-IL concentration enhanced from 0% to 100% (v/v) (FIG. 2C). Moreover, the adhesive strength of the engineered GelMA/Bio-IL cardiopatches was significantly higher than other synthesized cardiac sealants such as poloxamine-based hydrogels (˜17 kPa) (Cho, e. et al., 2012, Acta Biomater, 8(6):2223-32), and poly(glycerol sebacate)-co-lactic acid (24 kPa) (Chen, Q. et al., 2011, Soft Matter, 7(14):6484-6492), as well as commercial sealants such as Coseal™ and Evicel®. These results demonstrated that GelMA/Bio-IL cardiopatches can be readily applied to the surface of the myocardium and adhere strongly without the need for sutures or additional tissue adhesives.

The ability of GelMA/Bio-IL cardiopatches to seal tissue defects under applied pressure using collagen sheets was also evaluated based on a standard burst pressure test (Assmann, A. et al., 2017, Biomaterials, 140:115-127; Annabi, N. et al., 2017, Sci Transl Med, 9(410)) (FIG. 11C-FIG. 11D), as well as ex vivo explanted rat hearts (FIG. 2D-FIG. 2E). For burst pressure test using collagen sheets, small defects on sections of the tissue were created first, which were then sealed by photocrosslinking the cardiopatch on top of them. Then increasing air pressure was applied using a syringe pump connected to a pressure sensor until failure occurred (FIG. 11C). The results showed that the burst pressure of GelMA/Bio-IL cardiopatches adhered onto collagen sheets increased up to 5.36±1.01 kPa by increasing the concentration of Bio-IL (FIG. 11D). Moreover, the burst pressure of patches fabricated with 100% (v/v) Bio-IL was significantly higher than the burst pressure of both Coseal™ (1.7±0.1 kPa) and Evicel® (1.5±1.0 kPa) (FIG. 11D). The ex vivo burst pressure of explanted rat heart sealed with the adhesive patches fabricated with 10% (w/v) GelMA and varying concentrations of Bio-IL was also measured (FIG. 2D and FIG. 2E). For the ex vivo experiments, after sacrificing the animals, the blood vessels at the base of the heart were sealed with clamps, and a defect was created near the apex, which was then sealed with the patch by applying the GelMA/Bio-IL scaffold and photocrosslinking for 5 min. The results showed that failure occurred at a pressure of 29.97±5.56 kPa, 32.22±4.38 kPa, and 30.56±4.82 kPa for the heart sealed by the engineered cardiopatch containing 33%, 66%, and 100% (v/v) Bio-IL, respectively. The burst pressure of these cardiopatch formulations were similar to the pressure at failure for the intact heart (i.e., 31.05±0.67 kPa) (FIG. 2E). Furthermore, visual inspection revealed that failure did not occur due to detachment or rupture of the adhesive patches for all formulations, but due to bursting of the myocardium distal to the defect. The strong bonding of GelMA/Bio-IL cardiopatches to the myocardium was further confirmed via histological evaluation of the interface between the patch and the tissue (FIG. 2F). Hematoxylin and eosin (H&E) stained micrographs revealed a tight interlocking between the scaffold and the myocardium, which further demonstrated the intrinsic ability of the scaffolds to adhere strongly to the native tissue.

Standard wound closure (FIG. 2C) and burst pressure (FIG. 2E) tests demonstrated the high adhesive strength of the scaffolds, which was superior to commercial tissue adhesives, as well as other proposed bioadhesives, such as a hydrophobic light activated heart glue (Lang, N. et al., 2014, Sci Transl Med, 6(218): 218ra6-218ra6). These observations were further confirmed via histological evaluation, which revealed the tight interlocking at the interface between the patches and the myocardium (FIG. 2F). GelMA has been previously reported as a suitable material to obtain strong adhesion to wet tissues (Assmann, A. et al., 2017, Biomaterials, 140:115-127). This is due to the covalent bonds formed between methacrylate groups of GelMA and amine groups of tissue during photocrosslinking (Mooney, D. J. et al., 2007, Nat Mater, 6(5):327-328). Thus, once photocrosslinking has been completed, the anterior side of the cardiopatches will not adhere to the pericardial sac. In addition, GelMA/Bio-IL cardiopatches demonstrated significantly stronger adhesion to cardiac tissue when fabricated with higher concentrations of Bio-IL. This can be attributed to electrostatic interactions between the negatively charged surface of cardiac tissue (carboxyl group) and the positively charged choline-based Bio-IL (Li, J. et al., 2017, Science, 357(6349):378-381; Mehdizadeh, M. et al., 2013, Macromol Biosci, 13(3):271-288; Lawrence, P. G. et al., 2015, Langmuir, 31(4):1564-1574; Zhu, W. et al., 2018, Acta Biomat, 74:1-16). In fact, the strong adhesion between GelMA/Bio-IL patches and tissues can be attributed to the formation of two different types of chemical bonds: covalent bonds between GelMA and tissue, and ionic bonds between Bio-IL and tissue (schematic shown in FIG. 2F).

Recent studies have also reported the development of adhesive and conductive cardiac patches based on the incorporation of gold-nanorods (Malki, M. et al., 2018, Nano Lett) and dopamine (Liang, S. et al., 2018, Advanced Materials, 30(23)) in synthetic polymer networks. While these suture-free strategies greatly enhance the clinical translation of bioengineered cardiopatches by minimizing the risk of additional tissue damage, they may not lead to tissue repair and regeneration due to the absence of cell binding sites in the polymer network. In addition, previous groups have demonstrated the intrinsic potential of GelMA-based scaffolds to act as potent angiogenic niches (Kazemzadeh-Narbat, M. et al., 2017, Adv Health Mater, 6(10)). Therefore, in contrast to alternative strategies, GelMA/Bio-IL cardiopatches could also act as proangiogenic patches that could help salvage the ischemic myocardium during the early stages following MI (Cochain C. et al., 2013, Antioxid Redox Signal, 18(9):1100-1113). These scaffolds could also be used as a supportive layer that can minimize the risk of free wall rupture during the later stages of cardiac remodeling (Azevedo, P. S. et al., 2016, Arq Bras Cardiol, 106(1):62-69), owing to their strong tissue-adhesiveness biomimetic mechanical properties.

### Evaluation of GelMA/Bio-IL Cardiopatches Capability to Restore Impulse Propagation Across Severed Striated Muscle Ex Vivo

Electroconductive scaffolds could be used to restore electrical communication between excitable cell types to preserve the functionality of the tissue. Thus, the ability of GelMA/Bio-IL cardiopatches to restore impulse propagation between two pieces of skeletal muscle ex vivo was evaluated. For this, the rectus abdominis muscles of Wistar rats were explanted post-mortem, cut into square pieces, and placed 3 mm apart from each other on top of the scaffolds (FIG. 2G). Pulsed direct current test runs were conducted by applying 50 ms square pulses at increasing frequencies, using short platinum wires that were placed on one of the two samples. Muscle contraction was visually assessed on the opposite sample and the threshold voltage was recorded. As expected, the results showed that scaffolds containing higher concentrations of Bio-IL exhibited comparatively lower threshold voltages as compared to GelMA patches without Bio-IL (FIG. 2H). Therefore, the engineered GelMA/Bio-IL cardiopatches could be used to restore the propagation of electrical impulses and preserve the functionality of excitable tissues damaged by trauma or disease.

### In Vitro Contractile Activity and Phenotype of CMs Cultured on GelMA/Bio-IL Cardiopatches

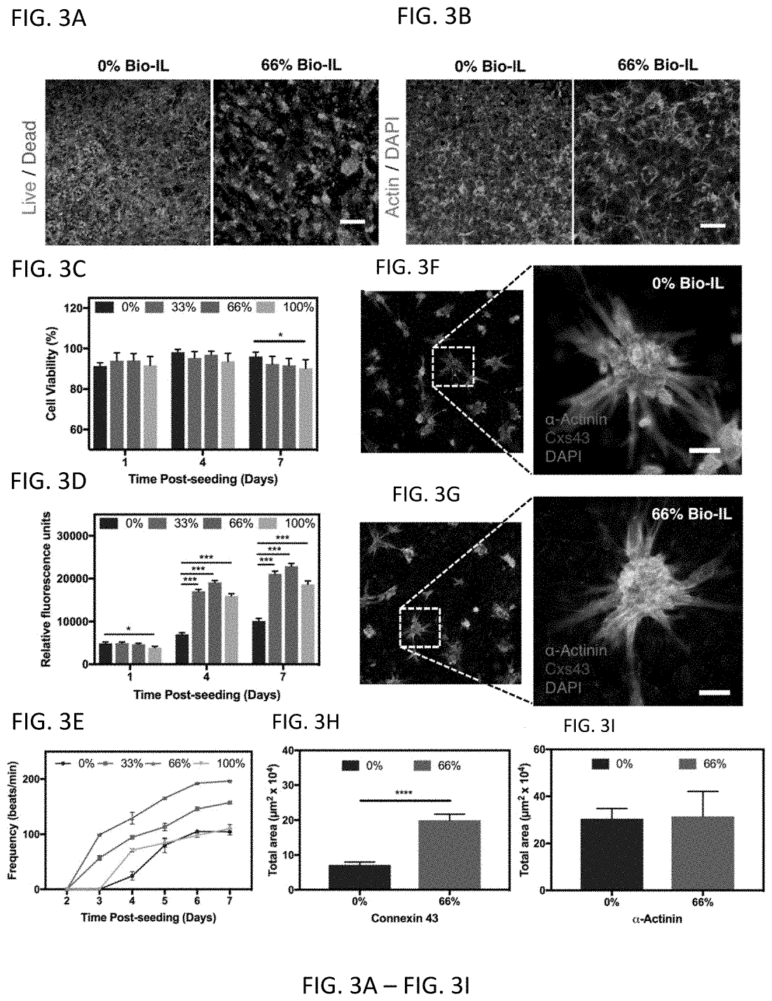

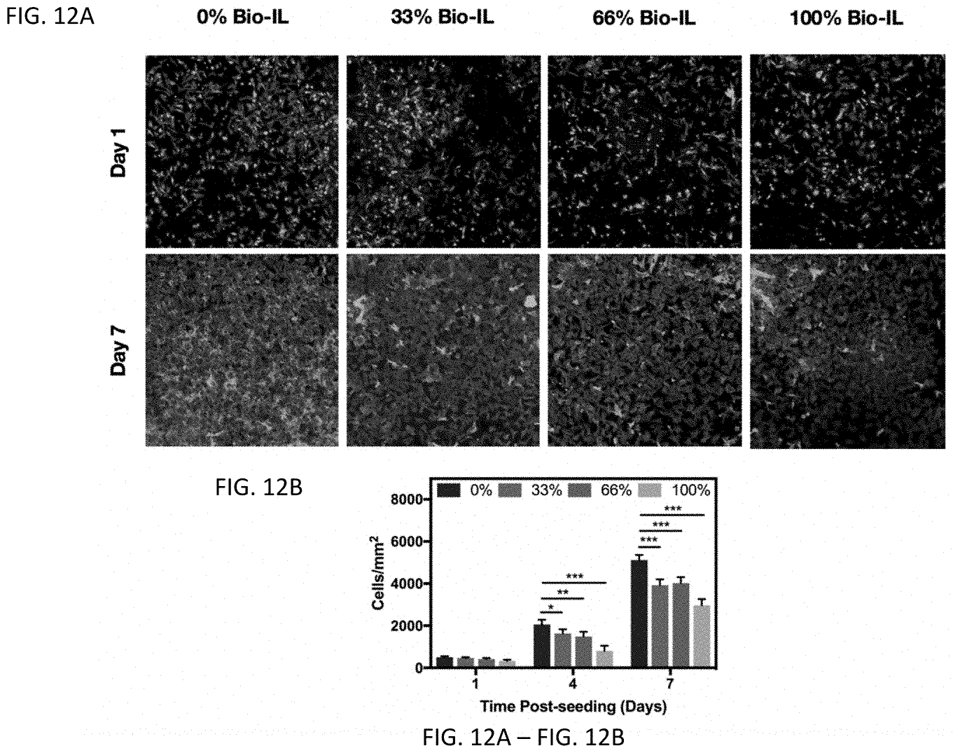

One of the most important aspects in the design of TE scaffolds is the accurate recapitulation of the different stimuli that modulate cell fate. CMs are electroactive cells that rely on electrical stimuli for maintaining tissue homeostasis and function (Liu, Y. et al., 2016, Mater Sci Eng C Mater Biol Appl, 69:865-874). Therefore, electroconductive scaffolds hold great potential for cardiac TE since they can promote the propagation of electrical impulses and enhance electromechanical coupling of CMs in vitro (Mathur, A. et al., 2016, Adv Drug Deliv Rev, 96:203-213). Here, the aim was to evaluate the ability of GelMA/Bio-IL cardiopatches to support the growth and the contractile function of co-cultures of freshly-isolated CMs and CFs. For this, primary CMs and CFs (2:1 ratio) were drop seeded on top of GelMA/Bio-IL scaffolds fabricated using different concentrations of Bio-IL. Cell viability and proliferation were evaluated using a commercial Live/Dead assay (FIG. 3A) and fluorescent F-actin/cell nuclei staining (FIG. 3B), respectively. The results demonstrated that the viability of CMs/CFs remained >90% up to day 7 post-seeding for all conditions tested (FIG. 3C). In addition, quantitative analysis of fluorescent images revealed that GelMA/Bio-IL scaffolds support the proliferation of CFs, which led to increasingly higher number of cells throughout the duration of the experiment (FIG. 12). The metabolic activity of cells growing on GelMA/Bio-IL cardiopatches was significantly higher than those growing on GelMA controls (FIG. 3D). The contractile activity of CMs seeded on GelMA/Bio-IL scaffolds was also evaluated. For this, cell-seeded scaffolds were imaged daily using an inverted microscope equipped with a CCD camera and a temperature-controlled chamber at 37° C. The beating frequency (beats/min, BPM) of the CMs was calculated from digitized video-recorded sequences using a custom MATLAB program. The results showed that cells grown on cardiopatches containing 33% and 66% (v/v) Bio-IL exhibited a comparatively more robust contractile behavior, when compared to pristine GelMA and GelMA with 100% (v/v) Bio-IL scaffolds (FIG. 3E). Moreover, cells grown on GelMA cardiopatches fabricated with 33% and 66% (v/v) Bio-IL exhibited observable contractility at day 7 post-seeding, and significantly higher beating frequencies (157.143±1.742 BPM and 196.524±1.018 BPM, respectively) than those growing on pristine GelMA patch and GelMA with 100% (v/v) Bio-IL cardiopatches (104.643±5.845 BPM and 110.210±7.360 BPM, respectively) (FIG. 3E). The lower contractile activity of the cells grown on GelMA patches can be due to the non-conductivity of the scaffold with no Bio-IL. On the other hand, when 100% (v/v) Bio-IL was used, the excess amount of Bio-IL might cover cell binding sites available on GelMA prepolymer, leading to lower cell attachment and contractile activity. Highest beating frequency was observed for the cells cultured on cardiopatches with 66% (v/v) Bio-IL.

The contractile function of the myocardium is established by a complex network of interconnected cells that communicate via gap junction proteins termed connexin, which mediate the propagation of electrical impulses (Stoppel, W. L. et al., 2016, Adv Drug Deliv Rev, 96:135-155). Here, the expression of phenotypic cardiac markers in cells grown on pristine GelMA scaffolds and GelMA cardiopatches containing 66% (v/v) Bio-IL, via immunofluorescent staining (IFS) against sarcomeric α-actinin (SAA) and connexin 43 (Cxs43) was evaluated. Representative fluorescent images revealed that cells seeded on the scaffolds self-organized in clusters of contracting CMs, which were attached to a layer of CFs proliferating on the surface of the scaffolds (FIG. 3F and FIG. 3G). The results also showed that cells grown on GelMA cardiopatches with 66% (v/v) Bio-IL exhibited significantly higher levels of Cxs43 expression, located mainly between the borders of the CFs, as compared to pristine GelMA patches (FIG. 3H). These observations demonstrate that GelMA/Bio-IL cardiopatches could aid in the propagation of electrical impulses between isolated cells to enhance the tissue-level functionality and beating of cardiac constructs.

The native myocardium is an electroactive tissue that can transfer electrical impulses that enable the synchronous contraction of the CMs, which in turn carry out the pump function of the heart. The results demonstrated that GelMA/Bio-IL cardiopatches could effectively promote the growth and function (FIG. 3) of co-cultures of CMs and CFs in vitro. Bio-IL conjugation led to a comparatively better contractile profile than pristine GelMA scaffolds, as demonstrated by the increased metabolic activity (FIG. 3D) and enhanced beating frequency (FIG. 3E) observed for conductive cardiopatches. In the context of cardiac electrophysiology, the propagation of electrical impulses is mediated via connexin proteins such as Cxs43, which enable heterocellular electrical coupling between CMs and CFs (McArthur, L. et al., 2015, Biochem Soc Trans, 43(3):513-518; Kohl, P. et al., 2005, J Electrocardiol 38(4 Suppl):45-50). Moreover, previous studies have showed that Cxs43-mediated CF coupling in vitro could enable synchronous spontaneous contraction in isolated CMs located up to 300 μm apart (Gaudesius, G. et al., 2003, Circulation research, 93(5):421-428). In this regard, IFS (FIG. 3F and FIG. 3G) showed that CFs growing on conductive scaffolds exhibited significantly higher levels of expression of Cxs43 when compared to GelMA-controls (FIG. 3H). Moreover, positive fluorescence against this gap junction protein was mainly located to the borders between the proliferating layer of CFs (FIG. 3G). These observations demonstrated that the conductive properties of GelMA/Bio-IL scaffolds promoted the electromechanical coupling of isolated CMs through the upregulation of Cxs43 in CFs. Furthermore, the disruption of electrical communication between cardiac cells has been shown to contribute to the generation of arrhythmias in fibrotic hearts in vivo and hinder the contractile function of TE cardiac constructs (Gaudesius, G. et al., 2003, Circulation research, 93(5):421-428). Therefore, Bio-IL conjugation could be used to aid in the rapid propagation of electrical impulses across heterocellular TE scaffolds, and lead to enhanced tissue-level functionality both in vitro and in vivo.

### In Vivo Application of GelMA/Bio-IL Cardiopatches Using a Murine Model of MI

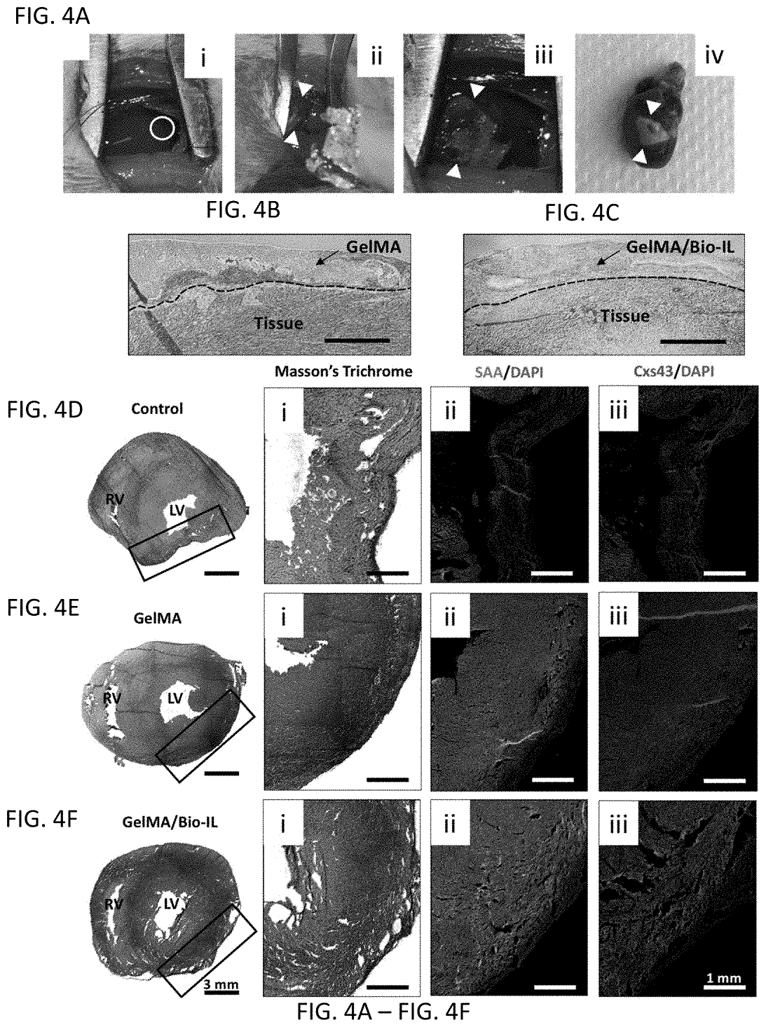

A series of structural and functional abnormalities occur after the onset of MI, which compromise the contractile function of the heart and could potentially lead to free wall rupture and death (Struthers, A. D. et al., 2005, Heart, 91 Suppl 2, ii14-6; discussion ii31, ii43-8). Electrospun fibrous patches have shown great potential to be used as cardio-supportive devices to help minimize the formation of non-contractile scar tissue and thinning of the infarcted myocardium (Zhao, G. et al., Adv Func Mater, 25(36):5726-5738; Prabhakaran, M. P. et al., 2011, Biomed Mater, 6(5):055001). However, the delivery of conductive scaffolds to the myocardium presents several risks that could potentially lead to impaired cardiac function or fatal arrhythmias (Cui, Z. et al., 2016, Engineering, 2(1):141-148). Thus, in this study, the feasibility, safety and in vivo functionality of GelMA/Bio-IL cardiopatches was evaluated using a murine model of MI via permanent ligation of the left anterior descending (LAD) coronary artery (FIG. 4A (i-iv)). All infarcts were confirmed via blanching of the myocardium distal to the site of ligation. Following the induction of MI, animals were divided into three treatment groups: sham, GelMA control, and GelMA/Bio-IL; and followed for a period of 3 weeks to allow for cardiac remodeling. Cardiopatches made of 10% (w/v) GelMA and 33% (v/v) Bio-IL were used for the treatment group that received conductive cardiopatches. At the end of this period, the animals were sacrificed, and the hearts were excised and processed for histological evaluation using Masson's trichrome and IFS against the cardiac markers SAA and Cxs43. The results showed strong adhesion of both GelMA (FIG. 4B) and GelMA/Bio-IL (FIG. 4C) patches to the myocardial tissue after 3 weeks. In addition, tissue ingrowth inside the scaffolds was observed for both GelMA and GelMA/Bio-IL samples. In addition, the cardiopatches were intact on the surface of the myocardium 21 days post implantation. Further, it was found that the sham controls exhibited significant thinning of the myocardium with a large aneurysmal section on the LV (FIG. 4D (i)). The infarcted area exhibited a marked reduction in the expression of both SAA and Cxs43, which was indicative of extensive CM death and the appearance of non-contractile scar tissue (FIG. 4D (ii-iii)). In contrast, the animals receiving both the GelMA (FIG. 4E (i)) and GelMA/Bio-IL (FIG. 4F (i)) cardiopatches showed comparatively less ventricular wall thinning and no apparent aneurysm at the site of MI after 3 weeks. Furthermore, the expression of phenotypic cardiac markers was maintained throughout the site of MI, which was indicative of the preservation of a fully functional myocardium for both hearts treated with GelMA scaffolds (FIG. 4E (ii-iii)) and GelMA/Bio-IL cardiopatches (FIG. 4F (ii-iii)). These results demonstrated that both GelMA-based patches could establish a cell-supportive microenvironment that prevented the remodeling of the myocardium at the site of MI and preserve normal tissue architecture. Furthermore, the expression of characteristic phenotypic markers SAA and Cxs43 was indicative of the preservation of viable myocardium and the maintenance of normal cardiac function.

Following MI, cardiac remodeling triggers a series of molecular and cellular changes that manifest clinically as changes in ventricular wall thickness and the appearance of fibrotic scar tissue (Azevedo, P. S. et al., 2016, Arq Bras Cardiol, 106(1):62-69). In recent years, electrospun scaffolds have shown great potential to be used as cardio-supportive devices, which can help minimize the formation of non-contractile scar tissue and thinning of the ventricular wall following MI (Prabhakaran, M. P. et al., 2011, Biomed Mater, 6(5):055001). Here, the feasibility and safety of in vivo delivery as well as the cardioprotective potential of GelMA/Bio-IL cardiopatches was evaluated using a murine model of MI via permanent LAD ligation (FIG. 4A (i-iv)). Histological evaluation revealed that the hearts treated with both GelMA and GelMA/Bio-IL patches exhibited minimal tissue remodeling and LV wall thinning, when compared to untreated animals (FIG. 4D-FIG. 4F). These observations demonstrated that the supportive function of both GelMA and GelMA/Bio-IL scaffolds could potentially ameliorate LV wall stress and preserve normal tissue architecture. These results were in accordance to previous studies showing that cardio-supportive devices with ECM-like properties can mediate endogenous repair mechanisms to improve heart function (Capulli, A. K. et al., 2016, Adv Drug Deliv Rev, 96:83-102). Moreover, these studies also showed that the attenuation of pathological cardiac remodeling occurred mainly due to architectural and compositional cues that potentiated tissue regeneration, independent of scaffold electroconductivity.

The results demonstrated that GelMA/Bio-IL scaffolds yielded tissue constructs with comparatively better in vitro functionality, which could be due in part to enhanced electromechanical coupling via upregulation of the gap junction protein Cxs43. Moreover, in vivo evaluation showed that both conductive and non-conductive GelMA-based scaffolds led to the preservation of normal tissue architecture by minimizing cardiac remodeling after MI. These observations could be explained in part due to the complex interplay of different bioactive cues that are normally present in vivo (Mauretti, A. et al., 2017, Stem Cells Int, 2017:7471582; Ebrahimi, B. et al., 2017, J Mol Cell Cardiol, 108:61-72; Safari, S. et al., 2016, Cell Mol Biol, 62(7):66-73; Pereira, M. J. et al., 2011, J Cardiovasc Transl Res, 4(5):616-630), which were not replicated in the in vitro experiments. Furthermore, these results demonstrated that cardiac remodeling could be effectively prevented using acellular scaffolds without the need for exogenous cytokines or growth factors, which is highly advantageous for the clinical translation of these scaffolds. For instance, Montgomery et al. recently reported a microfabricated injectable scaffold that could be used to deliver viable and functional CMs to the site of MI (Montgomery, M. et al., 2017, Nat Mater, 16(10):1038-1046). Although the scaffolds could be delivered through a minimally invasive procedure and significantly improved cardiac function following MI, both adult and stem cell-based strategies for the treatment of MI often shown highly heterogenous outcomes and poor clinical translation (Cambria, E. et al., 2016, Transfus Med Hemother, 43(4):275-281; Le, T. Y. et al., 2017, Heart Lung Circ, 26(4):316-322). Moreover, one of the most relevant characteristics of GelMA/Bio-IL cardiopatches was their high adhesive strength to the beating myocardium after photocrosslinking, even in the presence of blood (FIG. 4A (iii-iv)). Recently, Lang et al. reported the development of blood-resistant and light-activated surgical glue that could be used to seal cardiac wall defects in large animal models (Lang, N. et al., 2014, Sci Trans Med, 6(218):218ra6). Although this bioadhesive could be used by itself to form an on-demand hemostatic seal or in combination with a patch, GelMA/Bio-IL cardiopatches attached strongly to the native tissue without the need for additional adhesives or sutures. This also allowed the establishment of a tight interface and enhance the interlocking between GelMA and collagen fibers from the ECM-like fibrous patch and the myocardium, respectively, which is characteristic of strong tissue adhesives (Mandavi, A. et al., 2008, Proc Natl Acad Sci USA, 105(7):2307-2312; Artzi, N. et al., 2009, Adv Mater, 21(32-33):3399-3403). In addition, cardiopatches photocrosslinked on the surface of rat hearts did not exhibit significant changes in size due to water uptake of the scaffolds (FIG. 4A (iii-iv)). This demonstrates that swelling of the engineered cardiopatches would not significantly compress cardiac tissues in vivo.

In situ photocrosslinking of cardiopatches on the beating heart could possibly lead to systemic dissemination of unreacted components and thus, trigger toxic or inflammatory responses that could not be evaluated in vitro. The preliminary in vivo experiments confirmed that the electroconductive patches could be safely administered on the myocardium via in situ photopolymerization and did not induce any cytotoxicity. In addition, the heart is a highly dynamic organ and the presence of blood and other fluids, as well as cardiac beating could greatly impair the adherence of the patches to the myocardium in vivo. The in vivo results here demonstrated that the engineered patches exhibited high adhesion to the native murine myocardium without the need for suturing. Lastly, although the electroconductive and mechanical properties of the patches were tuned to mimic the native tissue, the delivery of a scaffold with these features to the myocardium could potentially impair cardiac function or even lead to fatal arrhythmias. Therefore, the current study aimed on evaluating the safety of in vivo delivery of Bio-IL functionalized patches before assessing the therapeutic effects of this strategy. The future study will focus on evaluating heart function after applying the electroconductive patches using echocardiography, as well as studying the molecular and cellular mechanisms that could be selectively triggered by the delivery of an electroconductive scaffold to the site of MI.

### Example 2: Engineering of a Conductive Cardiopatch Capable of Vasculogenesis and Stem Cell Homing for Cardiac Tissue Repair

While conductive cardiopatches may greatly benefit ischemic heart tissue, a drug delivery system composed of bioactive molecules to stimulate healing would be ideal to modulate meaningful tissue regeneration. Studies have shown that chemokines and growth factors present in the infarcted myocardium play an important role in healing and preserving overall heart function. Therefore, the aim is to further enhance cardiac tissue regeneration, by incorporating bioactive molecules inside the cardiopatches. Specifically, adding a drug delivery system to the conductive cardiopatches, which controls the release of stromal-cell derived factor 1 (SDF-1) and vascular endothelial growth factor (VEGF) directly to damaged cardiac tissues will be beneficial. Previous studies have shown that SDF-1 proteins are crucial for bone-marrow retention of haemopoietic stem cells and are involved in cardiogenesis, migration of primordial germ cells, and the recruitment of endothelial-cell progenitor cells to sites of ischemic cardiac tissue. For example, Naderi-Meshkin et al. has recently shown that the addition of SDF-1 into injectable hydrogels encouraged the site-directed homing and increased the retention of adipose tissue-derived mesenchymal stem cells (Askari et al., 2003, Lancet, 362:697-703). The incorporation of SDF-1 into the cardiopatches and optimize its release profile to recruit stem cells can aid in the repair of the myocardium following MI.

In addition, one drawback of traditional scaffolds used for cardiac tissue regeneration is their lack of a vascular network that exists in normal tissues. The formation of new blood vessels is essential to the healing of infarcted muscle tissue. Thus, there is a clear advantage to incorporating growth factors into biomaterial-based scaffolds for cardiac tissue engineering that will influence vasculogenesis. VEGF has been shown to be among the most powerful proangiogenic cytokines and has been associated with improvements in cardiac vascularization (Zacchigna, G. M., 2012, Gene Ther, 19:622-629). Co-delivery of VEGF and SDF-1 through the conductive cardiopatches will improve heart repair and promote cardiac vascularization.

The materials and methods employed in these experiments are now described.

Two type growth factors are loaded into conductive GelMA/Bio-IL cardiopatches: VEGF and SDF-1. The biochemical characteristics of both growth factors can be found in Table1.

TABLE 1
Biochemical characteristics of the cytokines applied
in the development of conductive cardiopatches.

| | Molecular | | | |
|---|---|---|---|---|
| | weight (kDa) | Size (nm) | Isoelectric point | Function |
| VEGF | 27.0 | ~10.0 | 9.2 | Angiogenesis |
| SDF-1 | 8.0 | ~4.0 | 9.8 | Stem cell |
| | | | | attraction |

Based on recent studies, the optimal pattern/timeline for the sustained release of SDF-1, in order to maximize its effect, is the initial 20-40% of local burst release followed by a sustained and steady release of the remaining 60% within one week (Zamproni, L. N> et al., 2017, J Pharm, 519:323-331). Regarding the VEGF, the sustained release of about 2-3 weeks after the burst release of 20% is considered optimum for angiogenesis in the infarcted cardiac tissue (Liu, G. et al., 2017, Biomaterials, 127:117-131).

To achieve these release profiles, two methods are used to incorporate VEGF and SDF-1 in the cardiopatches:

Method 1: Engineering Nanoparticles Loaded with VEGF

For controlled release of VEGF over 2-3 weeks, nanoparticles are engineered based on poly lactin-co-glycolic acid (PLGA) and poly lactin-co-glycolic acid-poly(ethylene glycol) methacrylate/succinimidyl-3-(2-pyridyldithio) propionate (PLGA-PEG-MA/SPDP) copolymers at ratio of 80:20 (Gholizadeh, S, et al., 2018, Inter J of Pharmaceuticals, 548:747-758). Different concentrations of VEGF are loaded into nanoparticles using a double emulsion technique (Oduk, Y. et al., 2018, Am J Physiol Heart Circ Physiol, 314:H278-H284). The freshly formulated nanoparticle suspension in Dulbecco's phosphate buffered saline (DPBS) are applied onto the cardiopatches.

### Chemically Conjugation of SDF-1 to Cardiopatches

For SDF-1 delivery, 1-ethyl-3-(3-dimethyl aminopropyl) carbodiimide/N-hydroxysuccinimide (EDC/NHS) coupling reactions are used for covalent bonding of SDF-1 to the GelMA fibrous mat to allow for sustained localized delivery of the SDF-1 (Fischer, M J E, 2010, Springer, 55-73). However, in order to obtain the initial burst release followed by a one-week sustained release, the solubilize SDF-1 in DPBS are directly loaded into the cardiopatches before photocrosslinking without any chemical bonding.

### Engineering Cardiopatches Containing Both VEGF and SDF-1

To fabricate angiogenic cardiopatches containing both growth factors, the electrospinning technique is used to develop GelMA fibrous mats. The GelMA mats are then soaked in a 1.25% Irgacure/ethanol solution. Mats are removed from the solution after 2 h. Solutions containing varying concentrations of Bio-IL (20, 25, 30%), SDF-1 (100-500 ng), and VEGF loaded nanoparticles (0.5-10 μg) in DPBS are also prepared. The fibrous GelMA mats are then placed in a mold followed by the addition of the Bio-IL/cytokine solutions. Cardiopatches are photocrosslinked via exposure to UV light for 300 sec. These patches are then be kept in a sterile environment until they are implanted in vivo.

### Method 2: Engineering GelMA Mats Containing VEGF and SDF-1

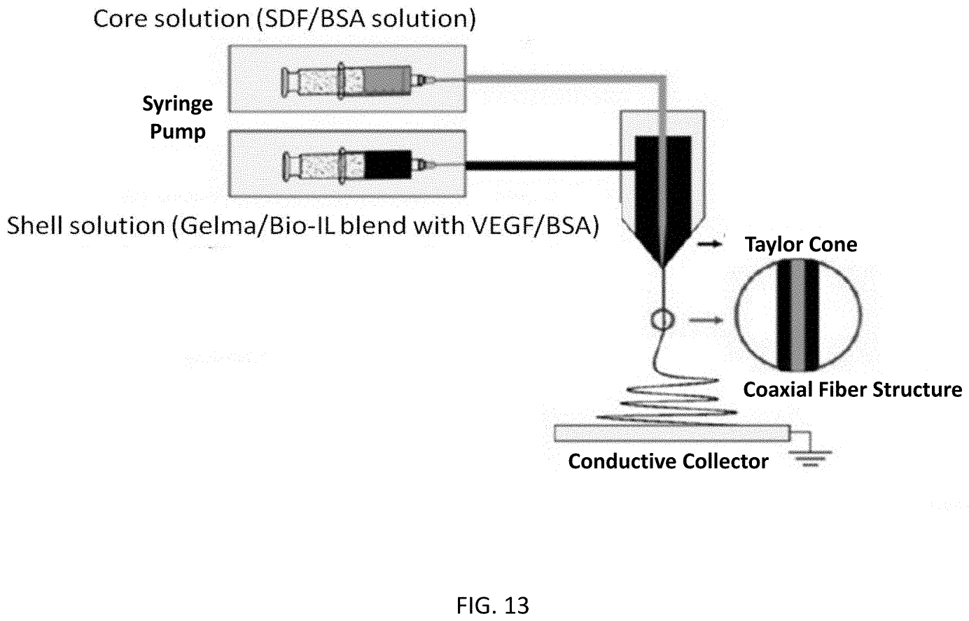

To control the release of VEGF and SDF-1, in the second method, a coaxial electrospinning approach is used to form shell containing VEGF and core containing SDF-1 (FIG. 13).

For VEGF loading in the sell, VEGF is blended with GelMA solution to form fibers with the diameter of 500 to 600 nm. For SDF-1 loading in the core, SDF-1 and bovine serum albumin (BSA) are added as a stabilizer. The addition of BSA will preserve the growth factor during the electrospinning process. In addition, it provides homogeneous protein distribution throughout the fibers, and SDF-1 can be delivered in a controlled manner due to the shell barrier which can elongate the release time and rate.

### Incorporating Bio-IL in the GelMA Mats Containing VEGF and SDF-1

The engineered GelMA mats are then soaked in a 1.25% Irgacure/ethanol solution. Mats are removed from the solution after 2 h and placed in a mold followed by the addition of the Bio-IL solutions. Cardiopatches are photocrosslinked via exposure to UV light for 300 seconds. These patches are then be kept in a sterile environment until they are implanted in vivo.

### Example 3: Study of the Function of Adhesive and Electroconductive Cardiopatches In Vivo Using a Murine MI Model

MI are stimulated in adolescent rats via 75 min of coronary artery ligation followed by reperfusion. Rats are divided into 5 groups based on the treatment they are receiving post-MI: (1) non-treatment group (control), (2) cardiopatches with no VEGF and SDF-1, (3) cardiopatches with an optimized concentration of VEGF (based on in vitro tests), (4) cardiopatches with an optimized concentration of SDF-1 (based on in vitro tests), and (5) cardiopatches with an optimized concentration of both VEGF and SDF-1.

The in vivo studies are performed for 6 weeks. The function of the heart is characterized by echocardiography on days 1, 14, 28, and 42. These results quantify the stroke volume, ejection fraction, cardiac output, and arterial elastance. Further, the infarct size and left ventricle wall thickness and compare these dimensions to the healthy heart to establish the occurrence of remodeling is evaluated. Further, the morphology of cardiac tissues using H&E and immunostaining is evaluated to determine if remodeling took place and if there was infiltration of inflammatory cell types into the myocardium. a significantly higher efficiency of heart function for animals treated with the conductive cardiopatches containing both VEGF and SDF-1AS compared to other treatment groups is expected. Also a higher level of blood vessel formation in the groups treated with VEGF is expected.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A biocompatible conductive scaffold comprising:
a fibrous biocompatible polymer conjugated to a first ionic constituent of a bio-ionic liquid (Bio-IL).

2. The scaffold of claim 1, wherein the first ionic constituent of a Bio-IL is an organic quaternary amine.

3. The scaffold of claim 2, wherein the organic quaternary amine is choline.

4. The scaffold of claim 1, wherein the polymer is selected from the group consisting of: gelatin, elastin, elastin like polypeptides (ELP), collagen, hyaluronic acid (HA), tropoelastin, chitosan, alginate, poly(glycerol sebacate) (PGS), poly(ethylene glycol) (PEG), and poly(lactic acid) (PLA).

5. The scaffold of claim 1, wherein the biocompatible polymer and the first ionic constituent are conjugated via a diacrylate linker.

6. The scaffold of claim 1, wherein the scaffold has a conductivity of at least about 0.23×10⁻¹±0.02×10⁻¹ siemens/meter (S/m).

7. The scaffold of claim 1, wherein the ratio of the biocompatible polymer to the first ionic constituent of a Bio-IL is from about 1:4 to about 4:1 on a weight basis.

8. The scaffold of claim 1, wherein the scaffold is capable of supporting cell proliferation, tissue organization, and/or a function of an excitable cell.

9. The scaffold of claim 8, wherein the cell is selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte.

10. The scaffold of claim 1, wherein the scaffold is biodegradable.

11. The scaffold of claim 1, wherein the scaffold is seeded with a population of cells prior to implantation, the cells selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte.

12. A method of preparing a conductive scaffold, the method comprising the steps of:
providing an ionic constituent of a bio-ionic liquid (Bio-IL) and a polymer;
creating a fibrous mat using the polymer;
placing the fibrous mat in a vacuum to remove excess solvent;
placing the fibrous mat in a solution bath containing a photoinitiator;
placing Bio-IL on the surface of the fibrous mat; and
crosslinking the scaffold.

13. The method of claim 12, wherein the first ionic constituent of a Bio-IL is an organic quaternary amine.

14. The method of claim 12, wherein the organic quaternary amine is choline.

15. The method of claim 12, wherein the polymer is selected from the group consisting of: gelatin, elastin, elastin like polypeptides (ELP), collagen, hyaluronic acid (HA), tropoelastin, chitosan, alginate, poly(glycerol sebacate) (PGS), poly(ethylene glycol) (PEG), and poly(lactic acid) (PLA).

16. The method of claim 12, wherein the polymer and the first ionic constituent of a Bio-IL are conjugated via a diacrylate linker.

17. The method of claim 12, wherein the scaffold has a conductivity of at least about 0.23×10⁻¹±0.02×10⁻¹ siemens/meter (S/m).

18. The method of claim 12, wherein the ratio of the polymer to the first ionic constituent of a Bio-IL is from about 1:4 to about 4:1 on a weight basis.

19. The method of claim 12, wherein the scaffold is capable of supporting cell proliferation, tissue organization, and/or a function of an excitable cell.

20. The method of claim 19, wherein the cell is selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte.

21. The method of claim 12, wherein the scaffold is biodegradable.

22. The method of claim 12, wherein the crosslinking step is performed for between about 100 and 500 seconds.

23. The method of claim 12, wherein the crosslinking step is performed using UV irradiation or visible light.

24. The method of claim 12, wherein the crosslinking step is performed on both side of the scaffold.

25. The method of claim 12, wherein the method further comprises a step of seeding cells on the scaffold, the cells selected from the group consisting of: a nerve cell, a muscle cell, a cardiomyocyte, a fibroblast, a preosteoblast, an endothelial cell, a mesenchymal stem cell, a pluripotent stem cell, an embryonic stem cell, a hematopoietic stem cell, an adipose derived stem cell, a bone marrow derived stem cell, an osteocyte, an epithelial cell, or a neurocyte.
